(19) United States
(12) Patent Application Publication
(10) Pub. No.: US 2023/0270804 A1
(43) Pub. Date: Aug. 31, 2023

(54) ANTI-HYPERCHOLESTEROLEMIA COMPOSITION AND A METHOD OF MANUFACTURING THE SAME

(71) Applicant: NBI BIOSCIENCES PRIVATE LIMITED, Gurgaon Haryana (IN)

(72) Inventors: Atul KUMAR, Gurgaon Haryana (IN); Richa SHRIVASTAVA, Noida Uttar Pradesh (IN); Kamireddy KIRAN, Polireddy Palem, S.P. Nellore, Andhra Pr (IN)

(21) Appl. No.: 18/005,721

(22) PCT Filed: Jul. 14, 2021
(86) PCT No.: PCT/IN2021/050680

§ 371 Date: Jan. 17, 2023

(30) Foreign Application Priority Data
Jul. 16, 2020 (IN) 202011030460

(51) Int. Cl. A61K 36/185; A61P 3/06; A61K 36/062; A61K 31/01; A61K 36/068; A61K 31/59; A61K 31/375; A61K 31/355; A61K 31/07; A61K 31/122; A61K 33/30; A61K 33/06; A61K 33/26; A61K 33/04; A61K 31/12; A61K 31/353; A61K 31/05; A61K 31/366; A61K 31/365

(52) U.S. Cl. CPC A61K 36/185; A61P 3/06; A61K 36/062; A61K 31/01; A61K 36/068; A61K 31/59; A61K 31/375; A61K 31/355; A61K 31/07; A61K 31/122; A61K 33/30; A61K 33/06; A61K 33/26; A61K 33/04; A61K 31/12; A61K 31/353; A61K 31/05; A61K 31/366; A61K 31/365

(57) ABSTRACT

The invention relates to an anti-hypercholesterolemia composition and a method of manufacturing the composition. Accordingly, the present invention discloses the composition to lower the cholesterol formation and a method of manufacturing the same. The anti-hypercholesterolemia composition comprises Basella alba leaf extract in the range of 30 to 70 wt %, red yeast rice extract in the range of 5 to 50 wt %, squalene extract in the range of 1 to 30 wt %, Cordyceps extract in the range of 10 to 70 wt % and at least one nutraceutically or pharmaceutically acceptable excipient in the range of 0.01 to 50 wt %.

### FIELD OF THE INVENTION

The invention relates to an anti-hypercholesterolemia composition and a method of manufacturing the composition.

### BACKGROUND OF THE INVENTION

Cholesterol is a waxy, fat-like substance found in all the cells in one's body. Cholesterol is essential for the production of hormones, vitamin D and substances required for digestion. The cholesterol required for these functions is made by the body or obtained from different food sources, especially from animal sources, such as egg yolks, meat, and cheese. Broadly, cholesterol is divided in to 2 categories: the bad cholesterol (low-density lipoproteins—LDL) and the good cholesterol (high-density lipoproteins—HDL). Generally, LDL deposits in blood vessels thereby leading to the reduction in size of the blood vessel and straining the flow of oxygen-rich blood throughout the body. Moreover, LDL can also lead to the formation of blood clots, which often break loose and block the flow of blood, causing a heart attack or stroke. Whereas, HDL, transports cholesterol from other parts of the body back to the liver, from where it is removed from the body.

Although cholesterol is essential for the production of hormones, vitamin D, and substances required for digestion, however cholesterol in higher concentration combines with other substances in the blood to form plaque. Over a period of time, this plaque sticks to the walls of the arteries leading to coronary artery disease. Hence, lowering the cholesterol level is of utmost importance. Hypercholesterolemia is elevation in the blood cholesterol levels primarily due to concentration of cholesterol in the cells and plasma. This leads to many cardiovascular diseases like atherosclerosis, coronary heart disease, myocardial infarction and stroke.

There are many risk factors that can indicate a propensity to have high levels of cholesterol, such as unhealthy eating habits and lifestyle, lack of physical activity, genetics, age, weight and health conditions such as diabetes, smoking, gender, race and ethnicity. Primarily, cholesterol is managed either by medicines or through dietary modifications. In medicines, statins are generally the first line treatment for the management of hypercholesterolemia. Although statins are the first line of treatment, they have a considerable poor safety record and higher side effects. This causes reduction in the long-term adherence to statins. Other cholesterol lowering drugs have a range of side effects which includes Muscle pain and damage, liver damage, diabetes, confusion and memory loss.

Therefore, there is felt a need for a composition which is devoid of the above-mentioned side effects and also has an anti-hypercholesterolemic effect.

### SUMMARY OF THE INVENTION

Accordingly, the present invention in one aspect provides a composition to lower the cholesterol formation. The anti-hypercholesterolemia composition comprises Basella alba leaf extract in the range of 30 to 70 wt %, red yeast rice extract in the range of 5 to 50 wt %, squalene extract in the range of 1 to 30 wt %, Cordyceps extract in the range of 10 to 70 wt % and at least one nutraceutically or pharmaceutically acceptable excipient in the range of 0.01 to 50 wt %.

The present invention in another aspect also discloses a method for manufacturing an anti-hypercholesterolemia composition.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

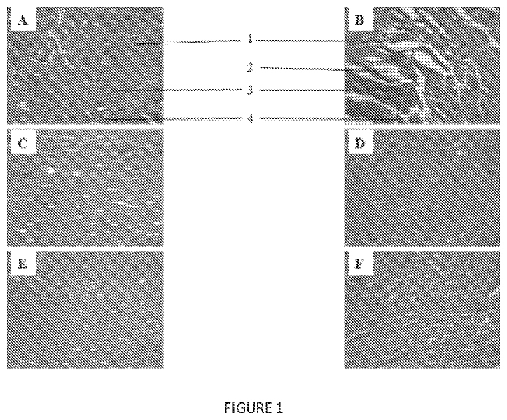

FIG. 1 shows histopathological images of the heart wherein:
1 depicts nuclei, 2 depicts fat cells, 3 depicts intercalated discs and 4 depicts cardiac muscles; and
A. Normal Control;
B. High Fat Diet (HFD) 1 ml/d+Normal Diet;
C. High Fat Diet (HFD)+Normal Diet+the composition of the present invention (OD);
D. High Fat Diet (HFD)+Normal Diet+the composition of the present invention (BD);
E. High Fat Diet (HFD)+Normal Diet+Statin; and
F. the composition of the present invention (OD) fed with normal diet in accordance with an embodiment of the invention.

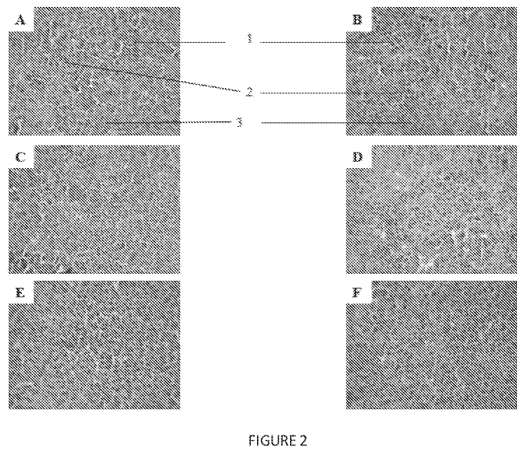

FIG. 2 shows a histopathological image of the spleen wherein:
1 depicts trabecula, 2 depicts white pulp and 3 depicts red pulp; and
A. Normal Control.
B. High Fat Diet (HFD) 1 ml/d+Normal Diet.
C. High Fat Diet (HFD)+Normal Diet+the composition of the present invention (OD);
D. High Fat Diet (HFD)+Normal Diet+the composition of the present invention (BD);
E. High Fat Diet (HFD)+Normal Diet+Statin; and
F. the composition of the present invention (OD) fed with normal diet in accordance with an embodiment of the invention.

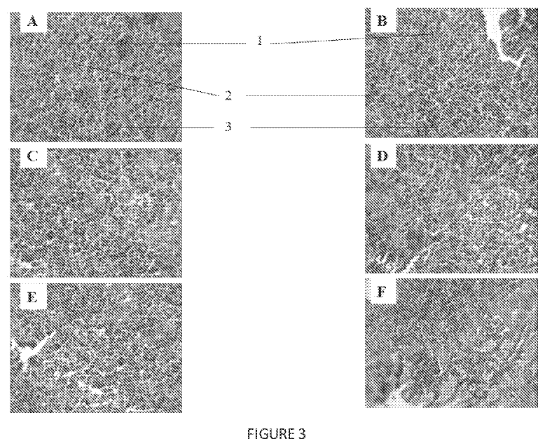

FIG. 3 shows a histopathological image of the lungs wherein:
1 depicts bronchiole, 2 depicts alveoli and 3 depicts veins; and
A. Normal Control;
B. High Fat Diet (HFD) lml/d+Normal Diet;
C. High Fat Diet (HFD)+Normal Diet+the composition of the present invention (OD);
D. High Fat Diet (HFD)+Normal Diet+the composition of the present invention (BD);
E. High Fat Diet (HFD)+Normal Diet+Statin; and
F. the composition of the present invention (OD) fed with normal diet in accordance with an embodiment of the invention.

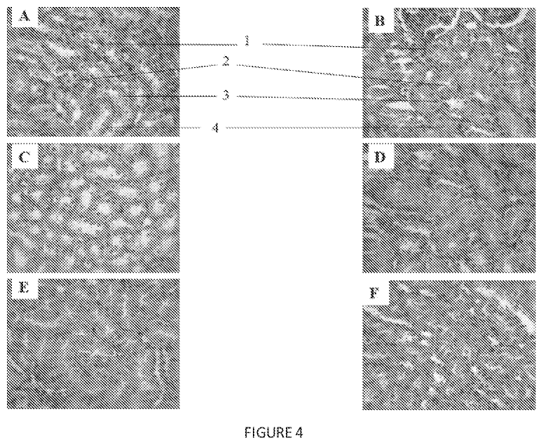

FIG. 4 shows histopathological images of the kidney wherein:
1 depicts macula densa, 2 depicts proximal tubules, 3 depicts capillaries and 4 depicts mesaglial cells; and
A. Normal Control;
B. High Fat Diet (HFD) 1 ml/d+Normal Diet;
C. High Fat Diet (HFD)+Normal Diet+the composition of the present invention (OD);
D. High Fat Diet (HFD)+Normal Diet+the composition of the present invention (BD);
E. High Fat Diet (HFD)+Normal Diet+Statin; and
F. the composition of the present invention (OD) fed with normal diet in accordance with an embodiment of the invention.

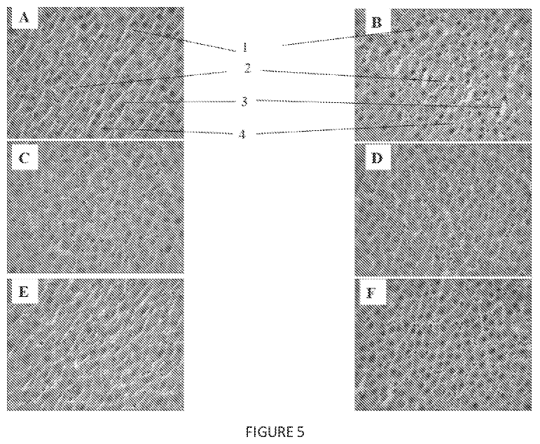

FIG. 5 shows histopathological images of the liver wherein:
1 depicts artery; 2 depicts bile duct; 3 depicts portal triad and 4 depicts portal vein; and
A. Normal Control.
B. High Fat Diet (HFD) 1 ml/d+Normal Diet;
C. High Fat Diet (HFD)+Normal Diet+the composition of the present invention (OD);
D. High Fat Diet (HFD)+Normal Diet+the composition of the present invention (BD);
E. High Fat Diet (HFD)+Normal Diet+Statin; and
F. The composition of the present invention (OD) fed with normal diet in accordance with an embodiment of the invention.

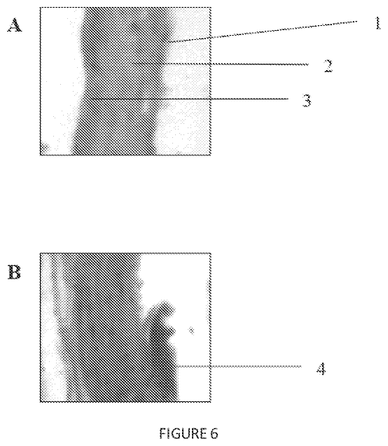

FIG. 6 shows histopathological images of Aorta in:
1 depicts tunica intima, 2 depicts tunica media, 3 depicts tunica adventitia and 4 depicts plaque; and
A. Normal Rat; and
B. HED diet rat showing change in the tunica intima having plaque formation in the cholesterol induced groups in accordance with an embodiment of the invention.

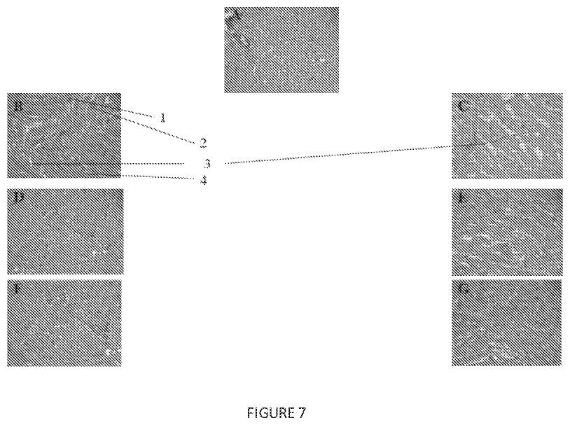

FIG. 7 shows histopathological images of Heart wherein:
1 depicts intercalated discs, 2 depicts nuclei, 3 depicts fat cells and 4 depicts cardiac muscles; and
A. Normal Control;
B. High Fat Diet (HFD) Discontinued;
C. High Fat Diet (HFD) Continued;
D. Statin+High Fat Diet (HFD) (Discontinued);
E. Statin+High Fat Diet (HFD) (Continued);
F. The composition of the present invention (BD)+High Fat Diet (HFD) (Discontinued); and
G. The composition of the present invention (BD)+High Fat Diet (HFD)
(Continued) in accordance with an embodiment of the invention.

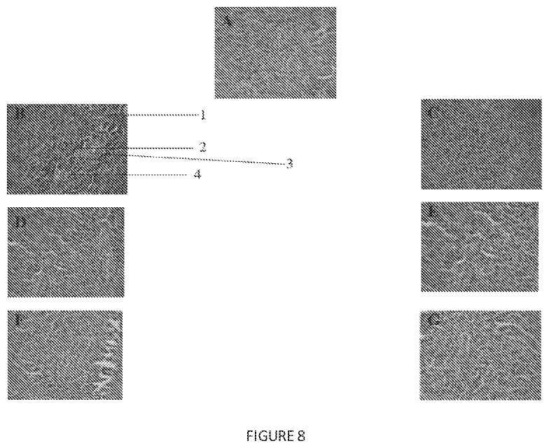

FIG. 8 shows histopathological images of spleen wherein:
1 depicts trabecula, 2 depicts white pulp, 3 depicts central arteiole, and 4 depicts red pulp; and
A. Normal Control.
B. High Fat Diet (HFD) Discontinued;
C. High Fat Diet (HFD) Continued;
D. Statin+High Fat Diet (HFD) (Discontinued);
E. Statin+High Fat Diet (HFD) (Continued);
F. The composition of the present invention (BD)+High Fat Diet (HFD) (Discontinued); and
G. The composition of the present invention (BD)+High Fat Diet (HFD)
(Continued) in accordance with an embodiment of the invention.

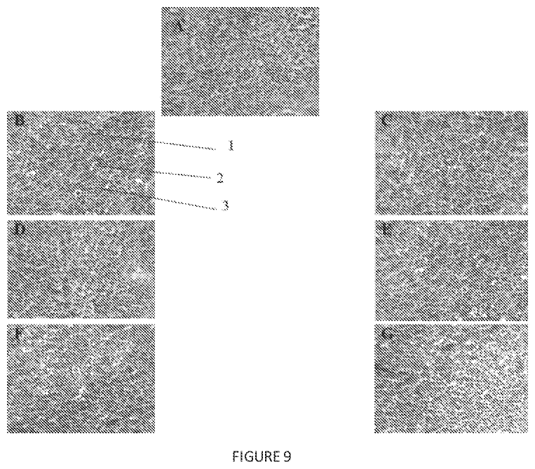

FIG. 9 shows a histopathological image of the lungs wherein:
1 depicts bronchiole, 2 depicts alveoli and 3 depicts veins; and
A. Normal Control;
B. High Fat Diet (HFD) Discontinued;
C. High Fat Diet (HFD) Continued;
D. Statin+High Fat Diet (HFD) (Discontinued);
E. Statin+High Fat Diet (HFD) (Continued);
F. The composition of the present invention (BD)+High Fat Diet (HFD) (Discontinued);
G. The composition of the present invention (BD)+High Fat Diet (HFD) (Continued) in accordance with an embodiment of the invention.

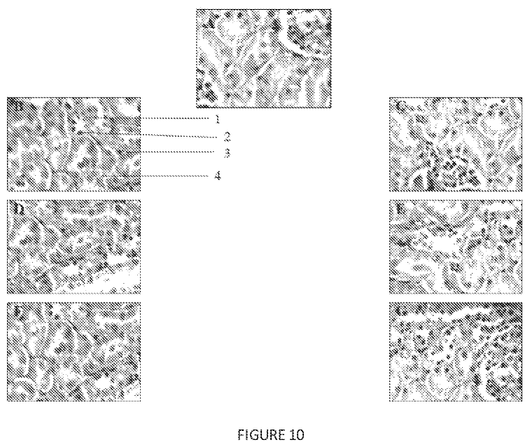

FIG. 10 shows histopathological images of the kidney wherein:
1 depicts macula densa, 2 depicts proximal tubules, 3 depicts capillaries and 4 depicts mesaglial cells; and
A. Normal Control;
B. High Fat Diet (HFD) Discontinued;
C. High Fat Diet (HFD) Continued;
D. Statin+High Fat Diet (HFD) (Discontinued);
E. Statin+High Fat Diet (HFD) (Continued);
F. The composition of the present invention (BD)+High Fat Diet (HFD) (Discontinued);
G. The composition of the present invention (BD)+High Fat Diet (HFD) (Continued) in accordance with an embodiment of the invention.

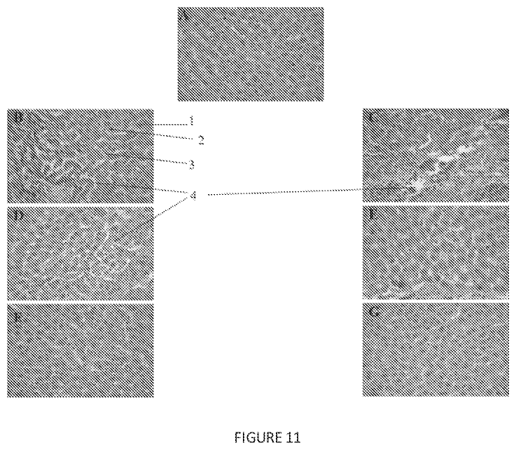

FIG. 11 shows histopathological images of the liver wherein:
1 depicts artery; 2 depicts bile duct; 3 depicts portal vein and 4 depicts portal triad; and
A. Normal Control.
B. High Fat Diet (HFD) Discontinued;
C. High Fat Diet (HFD) Continued;
D. Statin+High Fat Diet (HFD) (Discontinued);
E. Statin+High Fat Diet (HFD) (Continued);
F. The composition of the present invention (BD)+High Fat Diet (HFD) (Discontinued);
G. The composition of the present invention (BD)+High Fat Diet (HFD) (Continued) in accordance with an embodiment of the invention.

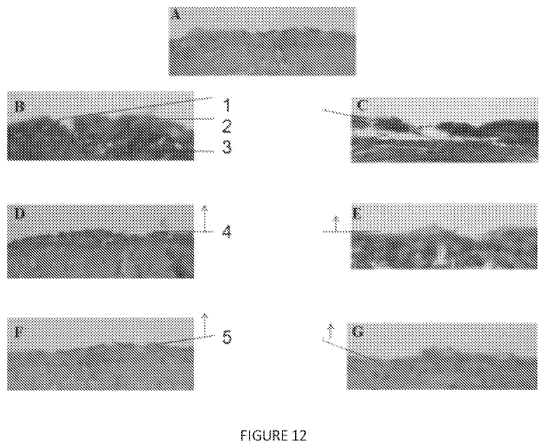

FIG. 12 shows histopathological images of Aorta wherein:
1 depicts plaque, 2 depicts tunica intima, 3 depicts tunica media, 4 depicts plaque dissolution and 5 depicts plaque recovered; and
A. Normal Control;
B. High Fat Diet (HFD) Discontinued;
C. High Fat Diet (HFD) Continued;
D. Statin+High Fat Diet (HFD) (Discontinued);
E. Statin+High Fat Diet (HFD) (Continued);
F. The composition of the present invention (BD)+High Fat Diet (HED) (Discontinued); and
G. The composition of the present invention (BD)+High Fat Diet (HFD) in accordance with an embodiment of the invention.

### DETAILED DESCRIPTION OF THE INVENTION

The present invention discloses a composition to lower the cholesterol formation and a method of manufacturing the same. In an aspect of the present invention disclosed is the anti-hypercholesterolemia composition. The composition comprises cholesterol lowering active ingredients and at least one nutraceutically or pharmaceutically acceptable excipient. In an embodiment of the present invention the cholesterol lowering active ingredients are selected from the extract(s), fraction(s), active compound(s) and phytochemical(s) or mixtures thereof from the group consisting of Basella alba, red yeast rice, Cordyceps and squalene. These ingredients are derived from conventional extractions procedures. In an exemplary embodiment of the present invention cholesterol lowering active ingredients are extracts or enriched fractions or pure compounds or the mixtures thereof.

Basella alba extract is obtained from Basella alba which is also known as Malabar spinach, vine spinach, and Ceylon spinach. It is native to the Indian subcontinent, Southeast Asia and New Guinea. However, it has been naturalized in China, tropical Africa, Brazil, Belize, Colombia, the West Indies, Fiji and French Polynesia, Accordingly, the extract or the raw materials can be sourced from any of these different natural sources. Typically, the plant extracts are derived from plant material selected from various parts of the plant such as, but not limited to, rhizomes, roots, stems, seeds, barks, flowers, leaves and fruits and is extracted using conventional extraction techniques using conventional solvents. In an embodiment of the present invention, Basella alba extract is derived from the leaves of Basella alba. In an embodiment of the present invention the Basella alba extract comprises polyphenols in an amount in the range of 0.5 to 2%, phenolics in an amount in the range of 0.5% to 2%, flavonoids in the range of 0.2% to 3% and ascorbic acid in the range of 0.2% to 1%.

The scope of the present invention is not only limited to Basella alba plant and products derived therefrom but also extends to botanically closely related plants specially belonging to same family, preferably belonging to same genus, still preferably belonging to same species having substantially similar phenotypic and genotypic characteristics.

The conventional solvents are selected from the group of, but not limited to, water, alcohol, organic solvent and a combination thereof or methods such as cryogenic extraction, maceration, infusion, decoction, percolation, hot continuous extraction (Soxhlet), aqueous alcoholic extraction, by fermentation, counter-current extraction, ultrasound extraction (sonication), Cold pressed extraction and supercritical fluid extraction whichever is suitable to obtain complete extract. The extracts may be in solid form or semi-solid form or liquid form or nano-emulsion form.

Red yeast rice also known as red rice koji, red fermented rice, red koji rice, anka or angkak. It is produced by the fermentation of cooked rice kernels with a Monascaceae mold, preferably Monascus purpureus, Monascus ruber, or Monascus pilosus. It also includes all the related strains of the given species such as Monascus purpureus went, Monascus purpureus NTU568, Monascus purpureus BCRC 31615, Monascus purpureus BCRC 31534, Monascus purpureus BCRC 31526, Monascus purpureus MTCC1090 which turns rice into reddish purple kernels due to its pigmentation capability. In an embodiment of the present invention the Monacolin K content in the red yeast rice extract is 0.1 ppm to 100 ppm and the Ankaflavin content in the red yeast rice extract is 0.2% to 5%.

The scope of the present invention is not only limited to Monascaceae mold and products derived therefrom but also extends to other microbiologically closely related organisms specially belonging to same family, preferably belonging to same genus, still preferably belonging to same species and strains having substantially similar phenotypic and genotypic characteristics.

Cordyceps also known as Chinese caterpillar fungus or Dong Chong Xia Cao or Caterpillar mushroom, Cs-4, Champignon chenille, Chinese caterpillar fungus, Ophiocordyceps, Tochukaso or Vegetable caterpillar. It is produced by fermentation process, grown on cooked rice kernels with Cordyceps culture, preferably Cordyceps sinensis and Cordyceps militaris which grows as a mat like structure on rice kernel medium or on liquid suspension by a fermentation process, known as mycelium and further this gives rise to a finger like fruiting body similar to mushrooms. Cordyceps sinensis and Cordyceps militaris are native to Tibet, Nepal and India. However, they are commonly spread throughout the Northern Hemisphere.

The scope of the present invention is not only limited to Cordyceps mold and products derived/extracted therefrom but also extends to other microbiologically closely related organisms specially belonging to same family, preferably belonging to same genus, still preferably belonging to same species and strains having substantially similar phenotypic and genotypic characteristics.

Squalene is the intermediate molecule in cholesterol biosynthesis, and is sourced from plant sources such as olive, soya bean, Amaranthus, rice bran, grape seed, almond, coconut, palm, wheat germ. The source is not only restricted to plants, but also includes animal sources such as shark, marine sources and of animal origin. In an embodiment of the present invention the squalene extract is commercially procured from the market.

In an embodiment of the present invention, the amount of Basella alba leaf extract is in the range of 30 to 70 wt %, the amount of red yeast rice extract is in the range of 5 to 50 wt %, the amount of squalene extract is in the range of 1 to 30 wt %, the amount of Cordyceps extract is in the range of 10 to 70 wt % and the amount of at least one nutraceutically or pharmaceutically acceptable excipient is in the range of 0.01 to 50 wt %.

In another embodiment of the present invention, the amount of Basella alba leaf extract is in the range of 35 to 55 wt %, the amount of red yeast rice extract is in the range of 5 to 20 wt %, the amount of squalene extract is in the range of 1 to 5 wt %, the amount of Cordyceps extract is in the range of 15 to 35 wt % and the amount of at least one nutraceutically or pharmaceutically acceptable excipient is in the range of 5 to 20 wt %.

In an embodiment of the present invention, at least one nutraceutically or pharmaceutically acceptable excipient is selected from the group consisting of at least one diluent, at least one super disintegrant, at least one binder, at least one lubricant, at least one glidant, at least one filler, at least one vitamin, at least one mineral, at least one phytochemical, at least one antioxidant and combinations thereof.

In an embodiment of the present invention, the at least one binder is selected from the group of Gelatin, ethyl cellulose, starch, Poly vinyl Pyrrolidone, Sodium alginate, carboxy methyl cellulose, Silicon oxide, Neusilin US2, Dextrin, Talc, Magnesium stearate, Aerosil and Micro crystalline cellulose.

In another embodiment of the present invention the at least one lubricant is selected from the group of Stearic acid, Calcium stearate, Sodium Benzoate and Polyethylene glycol.

In yet another embodiment of the present invention the at least one glidant is selected from the group of Corn starch and Silicon dioxide.

In still another embodiment of the present invention the at least one super disintegrant is selected from the group of Lactose, Micro crystalline cellulose and sorbitol.

In an embodiment of the present invention, the composition is prepared in the dosage forms selected from the group of a semi-solid mass, an oil and water soluble dispersion, nano emulsion, a capsule, tablet, syrup, a blend, a suspension and the like. In another embodiment, the dosage form is further selected from the group of immediate release, sustained release, and delayed release. In an exemplary embodiment of the present invention the composition of the present invention is encapsulated and is in a dosage form of a capsule. Depending upon the dosage the composition may comprise further excipients necessary for the manufacture of the preferred dosage form and its breakdown following ingestion and may be chosen by those skilled in the art.

In yet another embodiment of the present invention, the capsule shell is prepared using modified starch selected from the group consisting of, but not limited to, corn starch, gelatin, HPMC and carrageenan.

Advantageously, the composition of the present invention effectively lowers the cholesterol formation by lowering the HMG-CoA reductase enzyme and prevents LDL-Oxidation. The composition of the present invention also removes existing plaques and lowers the incidence of plaque formation and atherosclerosis incidence. Alternatively, or additionally, the composition may be for use in the management, treatment or prevention of hypercholesterolemia.

In an embodiment of the present invention, disclosed is an anti-hypercholesterolemia composition for the treatment or prevention of hypercholesterolemia and arteriosclerosis. The composition comprises Basella alba leaf extract is in the range of 30 to 70 wt %, red yeast rice extract is in the range of 5 to 50 wt %, squalene extract is in the range of 1 to 30 wt %, Cordyceps extract is in the range of 10 to 70 wt % and at least one nutraceutically or pharmaceutically acceptable excipient is in the range of 0.01 to 50 wt %.

In another aspect of the present invention, disclosed is a method for manufacturing the composition. The method initially involves obtaining the Basella alba leaf extract, Cordyceps extract red yeast rice extract and squalene extract. Pre-determined amounts of these extracts are then blended with pre-determined amounts of at least one nutraceutically or pharmaceutically acceptable excipient to obtain the anti-hypercholesterolemia composition of the present invention.

In an embodiment of the present invention, the Basella alba leaf extract is prepared by initially procuring a pre-determined amount of the Basella alba leaves followed by drying to remove the moisture. The dried leaves then undergo milling to obtain a milled plant material having a desired particle size suitable for maximum extraction. The milled plant material then undergoes extraction using different solvents through different extraction processes followed by removal and recovery of the solvent by rotary evaporator. The solvents used for the extraction are selected from the group of Ethanol, Hexane, Acetone, Double distilled water. The next step is nitrogen flushing which is carried out for removing the residual traces of the solvent to obtain a crude extract. The crude extract then undergoes winterization by dissolving the crude extract in a solvent followed by deep freezing. Typically, ethanol, methanol and hexane are used as solvents. The unwanted waxy material is removed by cold filtration followed by addition of pre-determined amounts of Aerosil and Dextrin to the crude extract and then the solvent is removed by rotary evaporation to obtain the Basella alba leaf extract in form of a dry powder. The powdered extract comprising total flavonoid content above 0.04% is stored at dry and non humid conditions.

In another embodiment of the present invention, the red yeast rice extract is prepared by initially grinding the red yeast rice to obtain a powder. The powdered red yeast rice is then subjected to extraction in a pulveriser or homogenizer using suitable solvents. The solvents are selected from the group of water, ethanol, acetone, methanol, Acetone, Di-ethyl ether and hexane. The solvent extraction as obtained undergoes filtration. Typically, the solvent extraction and filtration steps are repeated 2 to 5 times to achieve maximum extraction. The extract as obtained is kept undisturbed for 8 to 12 hours to obtain a red precipitate and a yellow supernatant. The yellow supernatant is then subjected to drying in a rotary evaporator to obtain a slurry. Drying excipients are then added to the slurry to obtain the extract of red yeast rice in powder form having the Monacolin K content in the range of 0.1 ppm to 100 ppm.

In yet another embodiment of the present invention, the Monascaceae mold extract is prepared by initially growing a pre-determined amount of the Monascus culture on rice kernels followed by drying to remove the moisture. The dried rice kernels fermented with Monascus culture then undergoes milling to obtain a milled mold material having a desired particle size suitable for maximum extraction. The powder of Monascus culture is extracted with acetone or ether or methanol or ethanol or dichloromethane or ethyl acetate or chloroform or hexane or water or in combinations of solvents. This process is repeated 3-5 times for maximum extraction. The extracted solvent is evaporated to dryness in vacuum drier or Rotary evaporator. The concentrated extract is passed through silica gel or sephadex or C18 columns to collect the pigmented fraction by eluting with suitable solvents or by its mixtures (acetone or ether or methanol or ethanol or dichloromethane or ethyl acetate or chloroform or hexane or water). To further purify the pigmented fraction a repeated step of column chromatography is adopted with the column material of silica or sephadex or C18, each fraction from the eluent is tested for monascin and ankaflavin content. Monascin and ankaflavin rich fraction is collected and further purification is carried out in preparative HPLC for high purity.

In an embodiment of the present invention, the squalene extract is procured directly from the vendors/market as a value-added product. The general procedure for purification of squalene from vegetable oil is silica-based column purification and elution with hexane.

In an embodiment of the present invention, the Cordyceps extract is prepared by initially growing a pre-determined amount of the Cordyceps culture in the form of mycelium or fruiting body grown on suitable medium. The fruiting bodies or mycelium are collected and dried to remove the moisture and powdered to the desired particle size. The whole powder or extracts are used in the prescribed quantity for formulation purpose.

In an embodiment the method of testing of purity of ingredients and quantification of active ingredients is carried out using spectrophotometer or Gas chromatography or Thin Layer Chromatography or High-Performance Thin layer chromatography or High-Performance Liquid Chromatography or Liquid chromatography and Mass spectrometry.

### EXAMPLES

### Example 1: Comparing the Anti-Atherosclerotic and Anti-Hypercholesteremia Potential of the Composition of the Present Invention with Statin (Atorvastatin)

Wistar strain albino rats of body weight ranging from 190-225 g were selected for the present study. The temperature and humidity were kept at optimum (22+2° C.; 40-70% and animals were exposed to natural chronological day night cycles. The experiments were carried out in conformity with the Institutional Animal Ethics Committee.

### Prophylactic Study:

The prophylactic study which lasted 90 days used 6 rats per group. The study was conducted to determine the anti-hypercholesteremic effect of the composition of the present invention with statin (Atorvastatin). The 6 groups were as follows:
1. Normal Control
2. High Fat Diet (HFD) 1 ml 1/d+Normal Diet
3. High Fat Diet (HFD)+Normal Diet+the composition of the present invention (OD)
4, High Fat Diet (HFD)+Normal Diet+the composition of the present invention (BD)
5. High Fat Diet (HFD)+Normal Diet+Statin
6. The composition of the present invention (OD) fed with normal diet
   Therapeutic study was done after inducing the cholesterol and atherosclerosis by HFD for 90 days and post 60 days treatment the efficacy of the composition of the present invention was investigated against statin. The body weights were recorded to the nearest gram (g) with proper tare to 0.0000 with the subject at steady state on the weighing machine. The same weighing machine was used for all the subjects. The machine was tested with a known set of weights for any error.

TABLE 1
Anthropometrical and Biochemical Data of Normal Control rats (Mean + S.D.)

| | Animal Group | | | | | | |
|---|---|---|---|---|---|---|---|
| | Normal Control | | | | | | |
| | Animal diet | | | | | | |
| | fed with normal diet | | | | | | |
| | Days | | | | | | |
| | 0 | 15 | 30 | 45 | 60 | 75 | 90 |
| | Days | Days | Days | Days | Days | days | days |
| Average | 195 ± 13.84 | 199.33 ± 26.15 | 199.16 ± 17.78 | 200.5 ± 25.29 | 210.8 ± 15.25 | 211.33 ± 9.02 | 213.33 ± 12.06 |
| Body | | | | | | | |
| Weight | | | | | | | |
| (gms) | | | | | | | |
| TC | 81.0 ± 1.56 | 82.0 ± 1.25 | 83.0 ± 2.0 | 83.16 ± 1.04 | 84.50 ± 2.29 | 86.03 ± 1.33 | 86.67 ± 2.06 |
| (mg/dl) | | | | | | | |
| TG | 16.9 ± 1.76 | 16.5 ± 1.26 | 16.3 ± 1.21 | 15.96 ± 1.56 | 16.23 ± 2.13 | 16.59 ± 0.45 | 16.16 ± 0.79 |
| (mg/dl) | | | | | | | |
| HDL-C | 34.6 ± 1.05 | 34.9 ± 1.15 | 34.66 ± 3.15 | 36.0 ± 2.64 | 36.9 ± 4.43 | 35.20 ± 0.30 | 35.33 ± 1.46 |
| (mg/dl) | | | | | | | |
| LDL-C | 46.4 ± 0.15 | 45.4 ± 0.65 | 44.66 ± 2.65 | 44.5 ± 2.29 | 45.46 ± 2.30 | 45.63 ± 0.67 | 46.20 ± 0.87 |
| (mg/dl) | | | | | | | |
| VLDL | 2.54 ± 0.12 | 2.41 ± 0.15 | 2.42 ± 0.07 | 2.45 ± 0.08 | 2.48 ± 0.09 | 3.20 ± 1.40 | 3.83 ± 1.05 |
| (mg/dl) | | | | | | | |
| WBC | 8 .99 ± 0.46 | 9.30 ± 0.56 | 8.99 ± 0.46 | 8.95 ± 0.42 | 8.86 ± 0.44 | 8.85 ± 0.11 | 8.91 ± 0.18 |
| (10³/ | | | | | | | |
| μl) | | | | | | | |
| RBC | 7.52 ± 0.18 | 7.62 ± 0.14 | 7.52 ± 0.18 | 7.40 ± 0.25 | 7.40 ± 0.46 | 7.54 ± 0.06 | 7.41 ± 0.18 |
| (10⁶/ | | | | | | | |
| μl) | | | | | | | |
| Hb | 12.86 ± 1.11 | 12.41 ± 1.51 | 12.88 ± 1.11 | 12.30 ± 1.05 | 12.30 ± 1.45 | 12.66 ± 0.09 | 12.96 ± 0.56 |
| (g/dl) | | | | | | | |
| PCV | 41.44 ± 1.08 | 41.87 ± 1.14 | 41.44 ± 1.08 | 42.30 ± 1.08 | 42.80 ± 1.81 | 41.96 ± 0.35 | 43.03 ± 1.31 |
| (%) | | | | | | | |
| Reticu- | 1.12 ± 0.09 | 1.14 ± 0.11 | 1.14 ± 0.08 | 1.10 ± 0.04 | 1.10 ± 0.50 | 1.19 ± 0.08 | 1.17 ± 0.08 |
| locyte | | | | | | | |
| (%) | | | | | | | |
| Platelet | 1048.19 ± 17.48 | 1051.28 ± 22.39 | 1065.19 ± 19.81 | 1060 ± 19.80 | 1085 ± 18.12 | 1076.00 ± 19.08 | 1070.00 ± 27.22 |
| count | | | | | | | |
| 10⁵/ | | | | | | | |
| mm3 | | | | | | | |
| Clotting | 111.01 ± 1.12 | 111.14 ± 1.9 | 115.01 ± 1.1 | 110 ± 1.08 | 112 ± 1.13 | 111.67 ± 2.52 | 113.67 ± 3.06 |
| time (Sec) | | | | | | | |
| Neutrophils | 26 | 25 | 25 | 26 | 26 | 25.57 | 25.93 |
| Lymphocyte | 71.4 | 72.4 | 72.4 | 71.35 | 71.35 | 71.53 | 71.5 |
| Monocyte | 1.45 | 1.58 | 1.35 | 1.45 | 1.5 | 1.51 | 1.51 |
| Eosinophil | 1.15 | 1.02 | 1.25 | 1.2 | 1.15 | 1.39 | 1.12 |

TABLE 2
Anthropometrical and Biochemical Data of HFD rats (Mean + S.D.)

| | Animal Group | | | | | | |
|---|---|---|---|---|---|---|---|
| | HFD | | | | | | |
| | Animal diet | | | | | | |
| | Fed with High fat diet (1 ml/day) along with normal diet | | | | | | |
| | Days | | | | | | |
| | 0 | 15 | 30 | 45 | 60 | 75 | 90 |
| | Days | Days | Days | Days | Days | days | Days |
| Average | 193.33 ± 11.9 | 207.5 ± 17.26 | 224.60 ± 14.61 | 241.83 ± 15.14 | 251.83 ± 15.76 | 263.33 ± 9.29 | 266.00 ± 7.00 |
| Body | | | | | | | |
| Weight | | | | | | | |
| (gms) | | | | | | | |
| TC | 82.50 ± 1.40 | 87.1 ± 2.19 | 108.3 ± 8.32 | 120.5 ± 4.92 | 127.13 ± 5.44 | 130.97 ± 6.44 | 137.33 ± 4.51 |
| (mg/dl) | | | | | | | |
| TG | 17.10 ± 1.25 | 19.2 ± 2.10 | 25.33 ± 2.08 | 31.83 ± 2.84 | 32.33 ± 0.70 | 34.60 ± 0.92 | 35.47 ± 0.81 |
| (mg/dl) | | | | | | | |
| HDL-C | 33.35 ± 1.10 | 30.0 ± 0.80 | 39.66 ± 3.21 | 36.5 ± 3.5 | 35.2 ± 3.2 | 34.03 ± 3.35 | 41.20 ± 6.41 |
| (mg/dl) | | | | | | | |
| LDL-C | 46.50 ± 0.50 | 49.87 ± 1.20 | 71.0 ± 5.56 | 81.33 ± 3.51 | 93.23 ± 1.86 | 91.93 ± 2.58 | 92.53 ± 2.10 |
| (mg/dl) | | | | | | | |
| VLDL | 2.50 ± 0.10 | 2.94 ± 0.40 | 3.43 ± 0.30 | 3.67 ± 0.02 | 3.90 ± 0.011 | 3.00 ± 0.78 | 3.60 ± 1.97 |
| (mg/dl) | | | | | | | |
| WBC | 9.10 ± 0.40 | 12.99 ± 0.46 | 9.10 ± 0.40 | 9.20 ± 0.40 | 8.40 ± 0.40 | 8.74 ± 0.16 | 8.75 ± 0.18 |
| (10³/ | | | | | | | |
| μl) | | | | | | | |
| RBC | 7.42 ± 0. | 6.42 ± 0.51 | 7.42 ± 0.26 | 7.20 ± 1.20 | 7.40 ± l. 40 | 7.62 ± 0.28 | 7.75 ± 0.15 |
| (10⁶/ | | | | | | | |
| μl) | | | | | | | |
| Hb | 12.16 ± 1.21 | 09.16 ± 1.31 | 12.16 ± 1.21 | 12.25 ± 1.20 | 12.37 ± 1.30 | 12.51 ± 0.25 | 12.49 ± 0.15 |
| (g/dl) | | | | | | | |
| PCV | 40.45 ± 1.18 | 34.24 ± 1.21 | 41.42 ± 1.10 | 41.00 ± 1.30 | 41.21 ± 1.10 | 42.22 ± 0.28 | 42.30 ± 0.36 |
| (%) | | | | | | | |
| Reticu- | 1.10 ± 0.10 | 1.08 ± 0.10 | 1.08 ± 0.15 | 1.05 ± 0.25 | 1.03 ± 0.33 | 1.22 ± 0.11 | 1.28 ± 0.08 |
| locyte | | | | | | | |
| (%) | | | | | | | |
| Platelet | 1050.20 ± 20.30 | 890.12 ± 21.42 | 1055.25 ± 18.05 | 1050.50 ± l5.50 | 1052.40 ± l4.40 | 1078.00 ± 21.17 | 1085.67 ± 12.22 |
| count | | | | | | | |
| 10⁵/ | | | | | | | |
| mm3 | | | | | | | |
| Clotting | 110.10 ± 1.15 | 112.15 ± 0.94 | 115.18 ± 1.10 | 112.18 ± 1.00 | 113.18 ± 1.00 | 115.10 ± 1.90 | 114.23 ± 1.91 |
| time (Sec) | | | | | | | |
| Neutrophils | 25 | 22.7 | 26 | 25 | 24 | 25.56 | 25.77 |
| Lymphocyte | 72.25 | 75.7 | 71.15 | 72.45 | 73.45 | 71.59 | 71.53 |
| Monocyte | 1.55 | 1.36 | 1.65 | 1.45 | 1.4 | 1.5 | 1.4 |
| Eosinophil | 1.2 | 1.24 | 1.2 | 1.1 | 1.15 | 1.35 | 1.3 |

TABLE 3
Anthropometrical and Biochemical Data of HFD + Composition of present invention (OD) rats (Mean + S.D.)

| | Animal Group | | | | | | |
|---|---|---|---|---|---|---|---|
| | HFD + Composition of present invention (OD) | | | | | | |
| | Animal diet | | | | | | |
| | Fed with high fat diet along with normal diet and treated once a day with NBI formulation | | | | | | |
| | Days | | | | | | |
| | 0 | 15 | 30 | 45 | 60 | 75 | 90 |
| | Days | Days | Days | Days | Days | Days | Days |
| Average | 203 ± 18.56 | 216.5 ± 19.30 | 225.83 ± 20.51 | 231.83 ± 20.93 | 223.16 ± 19.80 | 227.67 ± 4.51 | 236.67 ± 7.02 |
| Body | | | | | | | |
| Weight | | | | | | | |
| (gms) | | | | | | | |
| TC | 80.90 ± 0.50 | 88.74 ± 1.37 | 100.33 ± 5.03 | 109.66 ± 4.85 | 116.10 ± 6.08 | 118.67 ± 3.06 | 128.00 ± 5.57 |
| (mg/dl) | | | | | | | |
| TG | 16.10 ± 1.10 | 20.72 ± 1.74 | 26.66 ± 2.51 | 31.36 ± 4.06 | 34.20 ± 5.10 | 34.87 ± 0.95 | 34.33 ± 2.23 |
| (mg/dl) | | | | | | | |
| HDL-C | 33.05 ± 1.25 | 31.92 ± 1.15 | 34.33 ± 2.08 | 39.5 ± 4.76 | 41.4 ± 3.61 | 53.96 ± 4.66 | 58.90 ± 5.72 |
| (mg/dl) | | | | | | | |
| LDL-C | 45.25 ± 2.0 | 49.62 ± 0.22 | 60.0 ± 5.65 | 63.5 ± 1.5 | 65.26 ± 1.12 | 61.30 ± 6.19 | 65.70 ± 0.17 |
| (mg/dl) | | | | | | | |
| VLDL | 2.55 ± 0.14 | 3.14 ± 0.35 | 3.66 ± 0.14 | 3.69 ± 0.10 | 3.68 ± 0.04 | 3.41 ± 0.77 | 3.40 ± 2.03 |
| (mg/dl) | | | | | | | |
| WBC | 9.05 ± 0.45 | 10.18 ± 0.57 | 8.90 ± 0.55 | 9.10 ± 0.45 | 8.10 ± 0.46 | 8.93 ± 0.34 | 8.82 ± 0.15 |
| (10³/ | | | | | | | |
| μl) | | | | | | | |
| RBC | 7.25 ± 0.19 | 6.21 ± 0.19 | 7.25 ± 0.19 | 7.30 ± 0.25 | 7.35 ± 0.35 | 7.71 ± 0.42 | 7.64 ± 0.48 |
| (10⁶/ | | | | | | | |
| μl) | | | | | | | |
| Hb | 12.86 ± 1.11 | 08.52 ± 1.45 | 12.86 ± 1.11 | 13.05 ± l.05 | 13.07 ± l.07 | 12.71 ± 0.18 | 12.92 ± 0.12 |
| (g/dl) | | | | | | | |
| PCV | 43.04 ± 1.28 | 36.25 ± 1.19 | 42.90 ± 1.12 | 42.50 ± l.05 | 43.40 ± l.04 | 42.39 ± 0.59 | 42.25 ± 0.36 |
| (%) | | | | | | | |
| Reticu- | 1.13 ± 0.0 | 1.11 ± 0.14 | 1.10 ± 0.20 | 1.05 ± 0.25 | 1.08 ± 0.25 | 1.25 ± 0.09 | 1.31 ± 0.07 |
| locyte | | | | | | | |
| (%) | | | | | | | |
| Platelet | 1045.25 ± 22.31 | 919.22 ± 32.25 | 1051.20 ± 21.22 | 1060.22 ± 20.20 | 1065.55 ± 50.50 | 1073.00 ± 33.72 | 1059.33 ± 4.16 |
| count | | | | | | | |
| 10⁵/ | | | | | | | |
| mm3 | | | | | | | |
| Clotting | 111.50 ± 1.20 | 109.57 ± 0.89 | 111.50 ± 1.20 | 110.50 ± 1.30 | 110.40 ± 1.30 | 116.13 ± 1.06 | 118.70 ± 2.61 |
| time (Sec) | | | | | | | |
| Neutrophils | 24 | 22.9 | 25 | 26 | 26 | 25.96 | 26.62 |
| Lymphocyte | 73.5 | 74.4 | 72.6 | 71.55 | 71.55 | 71.57 | 70.93 |
| Monocyte | 1.3 | 1.44 | 1.2 | 1.25 | 1.25 | 1.18 | 1.18 |
| Eosinophil | 1.2 | 1.26 | 1.2 | 1.2 | 1.2 | 1.29 | 1.27 |

TABLE 4
Anthropometrical and Biochemical Data of HFD + Composition of present invention (BD) rats (Mean + S.D.)

| | Animal Group | | | | | | |
|---|---|---|---|---|---|---|---|
| | HFD + Composition of present invention (BD) | | | | | | |
| | Animal diet | | | | | | |
| | Fed with high fat diet along with normal diet and treated twice a day with NBI formulation | | | | | | |
| | Days | | | | | | |
| | 0 | 15 | 30 | 45 | 60 | 75 | 90 |
| | Days | Days | Days | Days | Days | Days | Days |
| Average | 211 ± 15.17 | 219.83 ± 15.02 | 201.83 ± 15.66 | 206.16 ± 24.19 | 200 ± 30.14 | 199.00 ± 4.58 | 207.67 ± 4.16 |
| Body | | | | | | | |
| Weight | | | | | | | |
| (gms) | | | | | | | |
| TC | 80.50 ± 0.90 | 86.92 ± 1.31 | 87.33 ± 4.50 | 90.0 ± 6.24 | 90.35 ± 7.91 | 86.33 ± 1.53 | 89.00 ± 1.00 |
| (mg/dl) | | | | | | | |
| TG | 16.45 ± 1.25 | 19.54 ± 1.78 | 22.33 ± 3.10 | 22.96 ± 2.23 | 23.46 ± 1.35 | 24.16 ± 3.72 | 24.20 ± 3.50 |
| (mg/dl) | | | | | | | |
| HDL-C | 34.50 ± 1.0 | 32.10 ± 1.16 | 37.0 ± 3.60 | 37.66 ± 2.30 | 37.57 ± 2.17 | 31.13 ± 1.25 | 33.73 ± 1.92 |
| (mg/dl) | | | | | | | |
| LDL-C | 46.10 ± 0.85 | 47.41 ± 0.15 | 57.66 ± 3.05 | 58.16 ± 6.5 | 57.23 ± 5.32 | 52.43 ± 1.99 | 52.18 ± 2.56 |
| (mg/dl) | | | | | | | |
| VLDL | 2.40 ± 0.10 | 3.10 ± 0.36 | 3.12 ± 0.07 | 3.06 ± 0.10 | 3.0 ± 0.16 | 2.77 ± 0.71 | 3.09 ± 0.85 |
| (mg/dl) | | | | | | | |
| WBC | 9.03 ± 0.3 | 9.50 ± 0.65 | 9.05 ± 0.45 | 9.10 ± 0.30 | 8.10 ± 0.30 | 8.62 ± 0.49 | 8.77 ± 0.31 |
| (10³/ | | | | | | | |
| μl) | | | | | | | |
| RBC | 7.15 ± 0.15 | 7.92 ± 0.14 | 7.15 ± 0.15 | 7.15 ± 0.40 | 7.35 ± 0.45 | 7.33 ± 0.26 | 7.34 ± 0.24 |
| (10⁶/ | | | | | | | |
| μl) | | | | | | | |
| Hb | 12.50 ± 1.05 | 09.59 ± 1.91 | 12.50 ± 1.05 | 14.30 ± 0.85 | 14.55 ± 0.87 | 14.05 ± 0.23 | 14.25 ± 0.38 |
| (g/dl) | | | | | | | |
| PCV | 42.40 ± 1.10 | 36.98 ± 1.13 | 41.42 ± 1.10 | 40.40 ± 2.20 | 41.20 ± 3.20 | 41.29 ± 0.38 | 41.39 ± 0.24 |
| (%) | | | | | | | |
| Reticu- | 1.05 ± 0.15 | 1.12 ± 0.18 | 1.08 ± 0.15 | 1.21 ± 0.20 | 1.41 ± 0.40 | 1.44 ± 0.25 | 1.44 ± 0.25 |
| locyte | | | | | | | |
| (%) | | | | | | | |
| Platelet | 1051.18 ± 17.30 | 930.24 ± 21.45 | 1055.25 ± 18.05 | 1069.18 ± 22.32 | 1068.18 ± 55.31 | 1074.33 ± 21.59 | 1078.33 ± 16.44 |
| count | | | | | | | |
| 10⁵/ | | | | | | | |
| mm3 | | | | | | | |
| Clotting | 110.01 ± 1.19 | 110.15 ± 0.84 | 115.18 ± 1.10 | 108.0 ± 1.10 | 105.0 ± 1.12 | 108.50 ± 2.59 | 109.87 ± 1.83 |
| time (Sec) | | | | | | | |
| Neutrophils | 26 | 23.8 | 26 | 24 | 24 | 25.4 | 25.4 |
| Lymphocyte | 71.4 | 73.8 | 71.15 | 73.25 | 73.2 | 72.07 | 72.18 |
| Monocyte | 1.5 | 1.3 | 1.65 | 1.6 | 1.55 | 1.11 | 1.11 |
| Eosinophil | 1.1 | 1.1 | 1.2 | 1.15 | 1.25 | 1.42 | 1.3 |

TABLE 5
Anthropometrical and Biochemical Data of HFD + Statin rats (Mean + S.D.)

| | Animal Group | | | | | | |
|---|---|---|---|---|---|---|---|
| | HFD + Statin | | | | | | |
| | Animal diet | | | | | | |
| | Fed with high fat diet along with normal diet and treated with Statin | | | | | | |
| | Days | | | | | | |
| | 0 | 15 | 30 | 45 | 60 | 75 | 90 |
| | Days | Days | Days | Days | Days | Days | Days |
| Average | 209.83 ± 26.39 | 213.16 ± 27.47 | 209.10 ± 20.10 | 223.83 ± 28.41 | 222.6 ± 17.46 | 223.05 ± 8.08 | 229.72 ± 6.13 |
| Body | | | | | | | |
| Weight | | | | | | | |
| (gms) | | | | | | | |
| TC | 81.95 ± 1.35 | 85.24 ± 1.55 | 85.66 ± 4.04 | 87.5 ± 5.04 | 88.5 ± 4.44 | 90.95 ± 2.05 | 90.22 ± 2.10 |
| (mg/dl) | | | | | | | |
| TG | 16.75 ± 1.10 | 18.9.33 ± 1.76 | 22.66 ± 3.10 | 24.86 ± 4.20 | 24.4 ± 4.61 | 22.67 ± 4.16 | 21.93 ± 3.90 |
| (mg/dl) | | | | | | | |
| HDL-C | 34.60 ± 1.50 | 31.56 ± 1.26 | 35.66 ± 2.08 | 38.33 ± 5.85 | 39.60 ± 5.57 | 33.53 ± 1.92 | 34.26 ± 1.26 |
| (mg/dl) | | | | | | | |
| LDL-C | 47.10 ± 1.25 | 48.68 ± 0.29 | 56.60 ± 4.50 | 57 ± 4.0 | 57.23 ± 4.43 | 55.27 ± 4.39 | 52.83 ± 1.76 |
| (mg/dl) | | | | | | | |
| VLDL | 2.48 ± 0.15 | 2.86 ± 0.35 | 2.96 ± 0.02 | 3.03 ± 0.19 | 3.0 ± 0.13 | 2.98 ± 0.84 | 3.12 ± 0.79 |
| (mg/dl) | | | | | | | |
| WBC | 8.90 ± 0.55 | 9.79 ± 0.57 | 9.03 ± 0.3 | 9.00 ± 0.50 | 8.00 ± 0.60 | 8.71 ± 0.23 | 8.76 ± 0.19 |
| (10³/ | | | | | | | |
| μl) | | | | | | | |
| RBC | 7.20 ± 0.20 | 7.20 ± 0.81 | 7.20 ± 0.20 | 7.05 ± 0.21 | 7.06 ± 0.31 | 7.36 ± 0.21 | 7.37 ± 0.20 |
| (10⁶/ | | | | | | | |
| μl) | | | | | | | |
| Hb | 11.95 ± 1.10 | 09.44 ± 1.19 | 11.9 ± 1.10 | 12.00 ± 1.20 | 12.20 ± 1.12 | 12.82 ± 0.25 | 12.78 ± 0.28 |
| (g/dl) | | | | | | | |
| PCV | 42.50 ± 1.80 | 35.85 ± 1.11 | 41.40 ± 1.15 | 42.10 ± 1.30 | 43.10 ± 1.30 | 41.40 ± 0.37 | 41.45 ± 0.29 |
| (%) | | | | | | | |
| Reticu- | 1.15 ± 0.05 | 1.10 ± 0.11 | 1.08 ± 0.10 | 1.25 ± 0.06 | 1.35 ± 0.06 | 1.70 ± 0.05 | 1.73 ± 0.02 |
| locyte | | | | | | | |
| (%) | | | | | | | |
| Platelet | 1040.19 ± 15.18 | 905.38 ± 21.23 | 1060.28 ± 23.30 | 1058.20 ± l6.82 | 1048.40 ± l6.84 | 1074.67 ± 22.94 | 1074.67 ± 22.94 |
| count | | | | | | | |
| 10⁵/ | | | | | | | |
| mm3 | | | | | | | |
| Clotting | 109.0 ± 1.10 | 107.45 ± 0.77 | 110.0 ± 1.0 | 114.0 ± 1.00 | 112.0 ± 1.05 | 110.81 ± 5.86 | 111.00 ± 5.57 |
| time (Sec) | | | | | | | |
| Neutrophils | 26 | 24.6 | 26 | 26 | 26 | 26.28 | 26.37 |
| Lymphocyte | 71.5 | 72.7 | 71.25 | 71.45 | 71.45 | 71.12 | 70.82 |
| Monocyte | 1.4 | 1.4 | 1.6 | 1.4 | 1.42 | 1.37 | 1.47 |
| Eosinophi1 | 1.1 | 1.3 | 1.15 | 1.15 | 1.13 | 1.22 | 1.35 |

TABLE 6
Anthropometrical and Biochemical Data of Formulation (OD) rats (Mean + S.D.)

| | Animal Group | | | | | | |
|---|---|---|---|---|---|---|---|
| | Invention of the present invention (OD) | | | | | | |
| | Animal diet | | | | | | |
| | Fed with normal diet and treated once a day with NBI formulation | | | | | | |
| | Days | | | | | | |
| | 0 | 15 | 30 | 45 | 60 | 75 | 90 |
| | Days | Days | Days | Days | Days | Days | Days |
| Average | 202.66 ± 19.97 | 206.33 ± 20.34 | 204.17 ± 17.16 | 200.33 ± 15.50 | 201 ± 20.97 | 202.33 ± 4.73 | 208.67 ± 3.51 |
| Body | | | | | | | |
| Weight | | | | | | | |
| (gms) | | | | | | | |
| TC | 82.25 ± 1.32 | 81.20 ± 0.50 | 81.0 ± 4.0 | 80.33 ± 5.85 | 82.03 ± 3.47 | 82.83 ± 4.25 | 83.53 ± 3.41 |
| (mg/dl) | | | | | | | |
| TG | 16.32 ± 1.05 | 14.5 ± 1.45 | 14.66 ± 1.52 | 14.10 ± 0.36 | 14.23 ± 0.50 | 15.60 ± 0.82 | 16.63 ± 1.26 |
| (mg/dl) | | | | | | | |
| HDL-C | 34.35 | 36.6 ± 1.05 | 35.66 ± 3.05 | 37.33 ± 3.21 | 39.0 ± 5.0 | 37.83 ± 1.45 | 37.83 ± 0.49 |
| (mg/dl) | | | | | | | |
| LDL-C | 46.25 ± 1.05 | 45.9 ± 0.10 | 43.66 ± 3.78 | 42.66 ± 3.05 | 42.66 ± 2.08 | 42.83 ± 3.52 | 42.13 ± 2.52 |
| (mg/dl) | | | | | | | |
| VLDL | 2.42 ± 0.05 | 2.50 ± 0.05 | 2.46 ± 0.60 | 2.40 ± 0.05 | 2.40 ± 0.55 | 2.17 ± 1.24 | 3.57 ± 1.63 |
| (mg/dl) | | | | | | | |
| WBC | 9.10 ± 0.15 | 9.45 ± 0.16 | 9.10 ± 0.15 | 9.05 ± 0.15 | 8.06 ± 0.16 | 8.63 ± 0.50 | 8.97 ± 0.35 |
| (10³/ | | | | | | | |
| μl) | | | | | | | |
| RBC | 7.05 ± 0.12 | 7.52 ± 0.21 | 7.05 ± 0.12 | 7.20 ± 0.30 | 7.30 ± 0.30 | 7.28 ± 0.22 | 7.39 ± 0.24 |
| (10⁶/ | | | | | | | |
| μl) | | | | | | | |
| Hb | 12.60 ± 1.15 | 12.91 ± 1.31 | 12.6 ± 1.15 | 14.10 ± 1.05 | 14.36 ± 1.07 | 13.75 ± 1.10 | 13.40 ± 0.53 |
| (g/dl) | | | | | | | |
| PCV | 41.80 ± 1.10 | 40.40 ± 1.10 | 40.85 ± 2.0 | 41.70 ± 2.05 | 40.51 ± 1.83 | 41.24 ± 0.26 | 41.38 ± 0.16 |
| (%) | | | | | | | |
| Reticu- | 1.10 ± 0.05 | 1.24 ± 0.05 | 1.15 ± 0.15 | 1.10 ± 0.10 | 1.12 ± 0.12 | 1.23 ± 0.09 | 1.25 ± 0.08 |
| locyte | | | | | | | |
| (%) | | | | | | | |
| Platelet | 1060.19 ± l9.10 | 1010.08 ± 21.30 | 1055.85 ± 15.10 | 1045.21 ± l4.12 | 1044.31 ± l2.13 | 1057.00 ± l4.00 | 1071.67 ± 21.55 |
| count | | | | | | | |
| 10⁵/ | | | | | | | |
| mm3 | | | | | | | |
| Clotting | 108.15 ± 1.32 | 101.10 ± 1.5 | 108.45 ± 1.0 | 109.25 ± 1.10 | 108.54 ± 1.10 | 107.93 ± 2.96 | 108.20 ± 2.60 |
| time (Sec) | | | | | | | |
| Neutrophils | 25 | 24 | 25 | 25 | 24 | 25.13 | 25.13 |
| Lymphocyte | 72.35 | 73.4 | 72.45 | 72.55 | 73.5 | 71.92 | 71.87 |
| Monocyte | 1.45 | 1.48 | 1.35 | 1.3 | 1.3 | 1.84 | 1.65 |
| Eosinophil | 1.2 | 1.12 | 1.2 | 1.15 | 1.2 | 1.11 | 1.35 |

Data presented in the table no. 1-6 as summarized in is the mean and standard deviation of lipid profile and hematological parameters that indicates that the composition of the present invention was able to maintain the body weight even after HFD treatment, whereas statin was not able to maintain the body weight. Further, the reduction in total cholesterol was similar with both groups (present composition and statin group) even after HFD diet treatment. However, significant inhibition was observed in total cholesterol synthesis (75%), LDL synthesis (80%) and triglycerides (50-70%). Apart from hemoglobin, 15% increase with the composition of the present invention twice a day, no significant changes were observed in blood parameters.

### Biochemical Assay

1.5-2 ml blood sample was taken from retino-orbital chamber in vials for biochemical estimations at every 15 days. Investigations performed included detailed Total Cholesterol—TC (mg/dl), Triglyceride—TG (mg/dl), High Density Lipoprotein—HDL-C (mg/dl), Low Density Lipoprotein—LDL-C (mg/dl), Very Low-Density Lipoprotein—VLDL (mg/dl), WBC (10³/u1), RBC (10⁶/u1), Hb (g/dl), PCV (%), Reticulocyte (%), Platelet count 10⁵/mm³, Clotting time (Sec), Neutrophils, Lymphocyte, Monocyte and Eosinophil. The estimations were done with Ecoline kits from Erba Mannheim, ERBA diagnostics Mannheml, Germany using Semi Autoanalyzer RX50 of MicroLabs Instruments and COUNCELL21 of Tulip Diagnostics Pvt. Ltd. Lipid peroxidation (Plasma & Liver), HMG Co-Reductase, Creatine phosphokinase (CPK), Lactate dehydrogenase (LDH), SGOT and SGPT were estimated at the end of both the studies using the kits.

### Assay of HMG CoA Reductase Activity:

The tissue homogenate was prepared by homogenizing 1 gm of tissue (liver) with 10 ml of saline arsenate solution. Further equal volumes of the fresh 10% tissue homogenate and diluted perchloric acid were mixed and allowed to stand for 5 minute and centrifuge (2000 rpm, 10 minutes). Then 1.0 ml of filtrate was treated with 0.5 ml of freshly prepared hydroxylamine reagent (alkaline hydroxylamine reagent in the case of HMG-CoA) and mixed. After 5 minutes, 1.5 ml of ferric chloride reagent as added to the same tube and shaken well. The readings were taken after 10 minutes at 540 nm vs. a similarly treated saline/arsenate blank.

TABLE 7
Biochemical enzyme estimation data of after sacrifice of rats (Mean + S.D.).

| | | | HFD + | HFD + | | |
|---|---|---|---|---|---|---|
| | | | Composition | Composition | | Composition |
| | | | of present | of present | | of present |
| | Normal | | invention | invention | HFD + | invention |
| Parameters | Control | HFD | (OD) | (BD) | Statin | (OD) |
| MDA Plasma | 33.50 ± 2.12 | 44.50 ± 1.41 | 35.13 ± 0.95 | 33.05 ± 0.90 | 35.10 ± 1.55 | 33.50 ± 1.38 |
| (nmol/ml) | | | | | | |
| MDA Liver | 77.00 ± 1.41 | 95.50 ± 2.12 | 76.33 ± 3.22 | 74.00 ± 0.83 | 80.60 ± 0.90 | 77.00 ± 1.55 |
| (nmol/g | | | | | | |
| tissue) | | | | | | |
| HMG Co | 208.00 ± 4.24 | 266.50 ± 2.83 | 220.42 ± 18.12 | 164.07 ± 4.06 | 163.92 ± 4.02 | 208.00 ± 2.76 |
| Reductase | | | | | | |
| (U/mg) | | | | | | |
| Creatine | 1.23 ± 0.04 | 1.34 ± 0.03 | 1.21 ± 0.06 | 1.12 ± 0.04 | 1.33 ± 0.04 | 1.21 ± 0.03 |
| phosphokinase | | | | | | |
| (U/ml) | | | | | | |
| LDH | 277.00 ± 4.24 | 318.50 ± 4.95 | 282.67 ± 12.39 | 277.83 ± 6.26 | 278.50 ± 5.50 | 278.67 ± 3.72 |
| (U/ml) | | | | | | |
| SGOT | 58.00 ± 1.41 | 75.50 ± 3.54 | 58.92 ± 2.20 | 56.92 ± 1.39 | 59.03 ± 1.49 | 57.33 ± 1.51 |
| (U/ml) | | | | | | |
| SGPT | 52.00 ± 1.41 | 69.50 ± 2.12 | 52.77 ± 1.11 | 50.50 ± 0.64 | 55.63 ± 0.51 | 51.50 ± 1.52 |
| (U/ml) | | | | | | |
| HbA1c | 3.65 ± 0.21 | 4.95 ± 0.21 | 3.59 ± 0.18 | 3.59 ± 0.13 | 3.72 ± 0.16 | 3.61 ± 0.16 |
| Liver | 3.30 ± 0.14 | 4.18 ± 0.11 | 3.44 ± 0.12 | 3.25 ± 0.09 | 3.90 ± 0.05 | 3.34 ± 0.12 |
| (g/100 g | | | | | | |
| bd · wt) | | | | | | |
| Brain | 1.33 ± 0.04 | 1.39 ± 0.01 | 1.31 ± 0.02 | 1.31 ± 0.03 | 1.30 ± 0.05 | 1.34 ± 0.03 |
| (g/100 g | | | | | | |
| bd · wt) | | | | | | |
| Heart | 2.77 ± 0.04 | 3.05 ± 0.07 | 2.76 ± 0.06 | 2.78 ± 0.05 | 2.79 ± 0.03 | 2.78 ± 0.03 |
| (g/100 g | | | | | | |
| bd · wt) | | | | | | |
| Kidney | 1.13 ± 0.04 | 1.18 ± 0.04 | 1.17 ± 0.02 | 1.14 ± 0.03 | 1.15 ± 0.03 | 1.14 ± 0.04 |
| (g/100 g | | | | | | |
| bd · wt) | | | | | | |
| Spleen | 0.62 ± 0.03 | 0.70 ± 0.03 | 0.64 ± 0.02 | 0.63 ± 0.03 | 0.65 ± 0.03 | 0.63 ± 0.03 |
| Lungs | 1.95 ± 0.04 | 2.08 ± 0.04 | 1.97 ± 0.02 | 1.95 ± 0.02 | 1.96 ± 0.02 | 1.96 ± 0.03 |
| (g/100 g | | | | | | |
| bd · wt) | | | | | | |
| Intestine | 3.50 ± 0.14 | 4.18 ± 0.04 | 3.53 ± 0.08 | 3.54 ± 0.05 | 3.54 ± 0.03 | 3.49 ± 0.09 |
| pancreas | 0.88 ± 0.04 | 0.91 ± 0.04 | 0.89 ± 0.04 | 0.91 ± 0.03 | 0.90 ± 0.05 | 0.91 ± 0.04 |
| (g/100 g | | | | | | |
| bd · wt) | | | | | | |

Data presented in table no 7 shows the enzyme estimations after the sacrifice of the animals. It is evident the HFD treatment induced the lipid peroxidation in liver by 20% which was significantly 23% reduced by the group that received the composition of the present invention twice a day and 15% by statin treatment. Creatine phosphokinase activity was found high in both HFD, and statin treated groups (15-20%) indicates the muscular damage. Inhibition in SGOT (24%) and SGPT (29%) activity was inhibited maximum in the group that received the composition of the present invention twice a day as compared to statin (15% and 20%). Both the composition of the present invention and statin treatment was able to maintain Blood glucose level similar to normal diet group even after HFD fed groups. Inhibition HMG Co reductase activity by the composition of the present invention and Statin is similar i.e., 35%.

### Statistical Analysis

Statistical analysis within the group and between the groups were analyzed between initial and final values using SPSS software. Mean+SD (Standard Deviation) of all clinical parameters and P-values were calculated by 2×2 contingency table using paired t-test. All the groups were compared by using Pearson's chi-square test. The data obtained were also analyzed by using analysis of variance (ANOVA) and multivariate analysis followed by Dunnett's multiple comparison test and Tukey Kramer test for determining the level of significance of the observed effects. All p values were two sided and differences were considered statistically significant for P<0.05; all significant data suggest the strength of association with clinical parameters. All the groups were compared by using Pearson's chi square test.

### Example 2: Histopathological Analysis

FIG. 1 depicts the histopathological images of heart. The histopathological finding of prophylactic study in heart significantly showed a clear deposition of fat in the cardiac muscles in HFD induced rats as compared to normal rat heart. While in case of statin the heart muscle showed relaxation in the cardiac muscles as the fat content is decreased. However, the rats treated with the composition of the present invention also showed the optimal recovery in cardiac muscle morphology even during the treatment with HFD. Conclusively, the composition of the present invention showed better results as compared within all groups.

FIG. 2 depicts the histopathological images of Spleen. The histopathological finding of prophylactic study in spleen indicated that there were no significant changes observed in any study groups. However, this indicates the safety of the composition of the present invention on spleen.

FIG. 3 depicts the histopathological images of Lungs. The histopathological finding of prophylactic study in lungs in different groups showed no pathological changes in any study group. However, this indicates the safety of the composition of the present invention on lungs.

FIG. 4 depicts the histopathological images of kidney. The histopathological finding of prophylactic study in kidney in different groups showed no pathological changes in any study group. However, this indicates the safety of the composition of the present invention on kidney.

FIG. 5 depicts the histopathological images of liver. The histopathological finding of prophylactic study in liver showed a significant formation of fatty cells (grade 2) in HFD induced rats. However, the intervention of the composition of the present invention at OD and BD dose levels showed decreasing order of fatty liver from grade 2 to grade 1 (evidenced by dilation in hepatic portal triad). In statin group, dilation of hepatic portal triad was more evident in comparison to the composition of the present invention (BD dose). It also indicates the safety of the composition of the present invention on liver. The composition of the present invention was also found useful to prevent the deposition of the fat bodies.

The experiments illustrate normal results and there is normal gain in the body weight. In High Fat Diet (HFD) 1 ml/d with Normal Diet group the body weight and lipid profile parameters showed significant increase as compared to the normal controls (P<0.005). In High Fat Diet (HFD) with Normal Diet and the composition of the present invention (OD) there was no major change recorded and the profile is almost similar to the group of High Fat Diet (HFD) 1 ml/d with Normal Diet. However, in High Fat Diet (HFD) with Normal Diet and the composition of the present invention (BD) showed drastic reversal in biochemical parameters as that in case of High Fat Diet (HFD) with Normal Diet with Statin group and showed significant association (P<0.005). Moreover, the composition of the present invention (OD) fed with normal diet also showed significant change in the biochemical profiling as compared to normal control group and found almost similar to the statin group. During Multivariate analysis the data suggest that the composition of the present invention (BD) showed improved effect as compared with the statin (P=0.070 and 0.058 respectively). Statistically the data showed significant association in biochemical and clinical profiling from day 0 to day 90 (P<0.001). Thus, the composition of the present invention demonstrates significant cholesterol lowering effect on anti-hypercholesterolemia induced Wistar rat's models even in the group i.e., continuous treatment of high fat diet and showed similar results as that of statin. Moreover, during the whole study the composition of the present invention at 82.5 mg/Kg body weight (OD & BD) oral dosage showed no sign of toxicity and mortality and found to be safe as per animal in house milieu.

FIG. 7 depicts the histopathological images of heart. The histopathological finding of therapeutic study in Heart of continued groups having on HFD diet showed morphological changes in the heart muscles (Fat deposition) as compared to normal rat heart. Intervention with the composition of the present invention showed a significant improvement in the HFD induced morphological changes in comparison to statin group. In the HFD-C group cardiac muscle were found stressed in the statin group due to high fat deposition whereas significant improvement was evident in the group provided with the composition of the present invention.
FIG. 8 depicts the histopathological images of spleen. The histopathological finding of therapeutic study in spleen in different groups showed no pathological changes in any study group thereby concluding that the composition of the present invention is safe on the spleen.
FIG. 9 depicts the histopathological images of lungs. The histopathological finding of therapeutic study in lungs in different groups showed no pathological changes in any study group thereby concluding that the composition of the present invention is safe on the lungs.
FIG. 10 depicts the histopathological images of kidney. The histopathological finding of therapeutic study in kidney in different groups showed no pathological changes in any study group thereby concluding that the composition of the present invention is safe on the kidney.
FIG. 11 depicts the histopathological images of liver. The histopathological finding of therapeutic study in liver showed a significant formation of fatty cells (grade 3) in HFD induced rats (continued), however the intervention of statin and the composition of the present invention showed more recovery in the group provided with the composition of the present invention (evidenced by dilation in hepatic portal triad) in comparison to the statin group. Moreover, the composition of the present invention improved the histopathology of fatty liver from grade 3 to grade 1.

### Example 3: Lipid Profile and Hematological Analysis

TABLE 7
Anthropometrical and Biochemical Data of Normal Control rats (Mean + ′S.D.)

| | Animal Group | | | | |
|---|---|---|---|---|---|
| | NORMAL CONTROL | | | | |
| | Days | | | | |
| Parameters | 0 | 15 | 30 | 45 | 60 |
| Average Body Weight | 221.00 ± 13.89 | 227.33 ± 12.74 | 231.33 ± 4.16 | 234.67 ± 4.93 | 243.17 ± 3.06 |
| TC (mg/dl) | 86.67 ± 2.52 | 87.00 ± 1.00 | 87.53 ± 0.31 | 89.03 ± 1.79 | 89.92 ± 1.89 |
| TG (mg/dl) | 16.67 ± 0.47 | 16.67 ± 0.76 | 16.67 ± 0.15 | 16.73 ± 0.21 | 16.90 ± 0.26 |
| HDL-C (mg/dl) | 35.17 ± 0.40 | 35.40 ± 0.75 | 35.50 ± 0.30 | 36.67 ± 0.61 | 37.03 ± 0.68 |
| LDL-C (mg/dl) | 46.17 ± 0.35 | 46.33 ± 0.61 | 46.73 ± 0.21 | 47.03 ± 0.21 | 47.50 ± 0.55 |
| VLDL (mg/dl) | 3.81 ± 0.04 | 3.72 ± 0.11 | 3.74 ± 0.02 | 3.74 ± 0.09 | 3.77 ± 0.09 |
| WBC(10³/μl) | 9.07 ± 0.14 | 9.02 ± 0.10 | 9.06 ± 0.03 | 9.05 ± 0.03 | 9.14 ± 0.10 |
| RBC(10⁶/μl) | 7.30 ± 0.10 | 7.43 ± 0.21 | 7.46 ± 0.13 | 7.49 ± 0.06 | 7.56 ± 0.10 |
| Hb (g/dl) | 12.91 ± 0.08 | 12.93 ± 0.15 | 12.97 ± 0.08 | 12.96 ± 0.07 | 13.09 ± 0.15 |
| PCV (%) | 43.08 ± 0.13 | 43.03 ± 0.21 | 43.06 ± 0.06 | 43.10 ± 0.09 | 43.53 ± 0.48 |
| Reticulocyte (%) | 1.17 ± 0.02 | 1.17 ± 0.01 | 1.17 ± 0.02 | 1.16 ± 0.03 | 1.17 ± 0.03 |
| Platelet count 10⁵/mm³ | 1076.67 ± 7.02 | 1079.00 ± 13.75 | 1073.33 ± 8.39 | 1073.00 ± 11.36 | 1083.50 ± 15.35 |
| Clotting time (Sec) | 113.33 ± 0.47 | 113.07 ± 0.38 | 113.09 ± 0.03 | 113.30 ± 0.44 | 114.43 ± 1.30 |
| Neutrophils | 25.91 | 25.63 | 25.71 | 25.8 | 26.06 |
| Lymphocyte | 71.53 | 71.38 | 71.73 | 71.68 | 71.4 |
| Monocyte | 1.44 | 1.38 | 1.37 | 1.3 | 1.31 |
| Eosinophil | 1.13 | 1.16 | 1.19 | 1.22 | 1.23 |

TABLE 8
Anthropometrical and Biochemical Data of HFD Discontinued rats (Mean ± S.D.)

| | Animal Group | | | | |
|---|---|---|---|---|---|
| | HFD Discontinued | | | | |
| | Days | | | | |
| Parameters | 0 | 15 | 30 | 45 | 60 |
| Average Body Weight | 272.67 ± 11.23 | 269.67 ± 5.03 | 264.00 ± 5.66 | 272.00 ± 8.49 | 270.33 ± 4.13 |
| TC (mg/dl) | 137.34 ± 3.51 | 131.67 ± 2.52 | 131.00 ± 8.49 | 133.0 ± 1.176 | 134.56 ± 1.75 |
| TG (mg/dl) | 38.33 ± 2.51 | 36.33 ± 2.08 | 35.50 ± 2.12 | 35.65 ± 0.21 | 36.05 ± 0.40 |
| HDL-C (mg/dl) | 41.67 ± 1.52 | 41.33 ± 2.52 | 39.50 ± 2.12 | 39.15 ± 0.49 | 39.31 ± 0.73 |
| LDL-C (mg/dl) | 92 ± 2.01 | 86.67 ± 2.08 | 85.00 ± 5.66 | 85.0 ± 0.28 | 85.92 ± 0.89 |
| VLDL (mg/dl) | 3.71 ± 0.27 | 3.83 ± 0.10 | 3.76 ± 0.05 | 3.75 ± 0.01 | 3.78 ± 0.04 |
| WBC(103/μl) | 8.75 ± 0.18 | 8.71 ± 0.07 | 8.65 ± 0.07 | 8.70 ± 0.07 | 8.79 ± 0.10 |
| RBC(10⁶/μl) | 7.98 ± 0.19 | 8.07 ± 0.10 | 8.13 ± 0.04 | 8.14 ± 0.03 | 8.21 ± 0.09 |
| Hb (g/dl) | 12.51 ± 0.22 | 12.55 ± 0.36 | 12.60 ± 0.21 | 12.79 ± 0.16 | 12.83 ± 0.25 |
| PCV (%) | 42.18 ± 0.72 | 41.83 ± 0.29 | 42.13 ± 0.04 | 42.17 ± 0.02 | 42.49 ± 0.47 |
| Reticulocyte (%) | 1.24 ± 0.02 | 1.26 ± 0.03 | 1.25 ± 0.07 | 1.27 ± 0.01 | 1.27 ± 0.03 |
| Platelet count 10⁵/mm³ | 1078 ± 16.82 | 1078.67 ± 19.50 | 1065.50 ± 20.51 | 10763 ± 7.94 | 1083.20 ± 12.11 |
| Clotting time (Sec) | 113.34 ± 1.15 | 113.23 ± 0.87 | 111.50 ± 4.95 | 112.10 ± 1.15 | 113.26 ± 1.80 |
| Neutrophils | 25.5 | 25.67 | 26 | 25.78 | 26.05 |
| Lymphocyte | 72 | 71.88 | 71.4 | 71.68 | 71.37 |
| Monocyte | 1.35 | 1.38 | 1.4 | 1.28 | 1.31 |
| Eosinophil | 1.25 | 1.25 | 1.2 | 1.25 | 1.27 |

TABLE 9
Anthropometrical and Biochemical Data of HFD continued rats (Mean + S.D.)

| | Animal Group | | | | |
|---|---|---|---|---|---|
| | HFD Continued | | | | |
| | Days | | | | |
| Parameters | 0 | 15 | 30 | 45 | 60 |
| Average | 270.34 ± 14.50 | 286.00 ± 10.58 | 293.00 ± 2.83 | 293.00 ± 7.07 | 292.60 ± 8.56 |
| Body Weight | | | | | |
| TC (mg/dl) | 137.33 ± 3.51 | 148.00 ± 6.00 | 158.50 ± 4.95 | 154.5 ± 0.71 | 147.38 ± 5.84 |
| TG (mg/dl) | 38 ± 2 | 42.33 ± 3.51 | 46.50 ± 2.12 | 43.05 ± 0.64 | 43.18 ± 0.49 |
| HDL-C | 42 ± 2.65 | 43.33 ± 1.53 | 46.00 ± 1.41 | 46.10 ± 0.42 | 44.13 ± 2.48 |
| (mg/dl) | | | | | |
| LDL-C | 92.34 ± 3.21 | 87.00 ± 2.00 | 93.00 ± 2.83 | 91.85 ± 0.92 | 95.22 ± 2.12 |
| (mg/dl) | | | | | |
| VLDL | 3.72 ± 0.18 | 3.63 ± 0.08 | 3.85 ± 0.14 | 3.67 ± 0.02 | 3.70 ± 0.04 |
| (mg/dl) | | | | | |
| WBC(103/μl) | 8.72 ± 0.23 | 8.68 ± 0.26 | 8.80 ± 0.07 | 9.0 ± 0.28 | 9.08 ± 0.19 |
| RBC(10⁶/μl) | 7.92 ± 0.08 | 7.98 ± 0.16 | 8.13 ± 0.04 | 8.09 ± 0.05 | 8.13 ± 0.05 |
| Hb (g/dl) | 12.52 ± 0.20 | 12.68 ± 0.18 | 12.65 ± 0.07 | 12.7 ± 0.14 | 12.70 ± 0.15 |
| PCV (%) | 42.28 ± 0.40 | 42.65 ± 0.62 | 43.55 ± 0.64 | 43.23 ± 0.04 | 43.63 ± 0.38 |
| Reticulocyte | 1.25 ± 0.04 | 1.26 ± 0.03 | 1.39 ± 0.05 | 1.29 ± 0.01 | 1.29 ± 0.02 |
| (%) | | | | | |
| Platelet | 1079 ± 8.49 | 1083.00 ± 6.56 | 1076.50 ± 12.02 | 1059 ± 6.36 | 1059.67 ± 10.11 |
| count | | | | | |
| 10⁵/mm³ | | | | | |
| Clotting time | 112.75 ± 0.35 | 114.67 ± 3.79 | 113.00 ± 1.41 | 112.6 ± 0.21 | 113.53 ± 0.80 |
| (Sec) | | | | | |
| Neutrophils | 25.93 | 25.67 | 25.5 | 25.85 | 25.53 |
| Lymphocyte | 71.5 | 71.5 | 72 | 71.63 | 71.88 |
| Monocyte | 1.3 | 1.36 | 1.3 | 1.3 | 1.34 |
| Eosinophil | 1.3 | 1.26 | 1.2 | 1.25 | 1.26 |

TABLE 10
Anthropometrical and Biochemical Data of Statin + HFD discontinued rats (Mean ± S.D.)

| | Animal Group | | | | |
|---|---|---|---|---|---|
| | Statin + HFD (Discontinued) | | | | |
| | Days | | | | |
| Parameters | 0 | 15 | 30 | 45 | 60 |
| Average | 270.33 ± 8.74 | 268.00 ± 8.89 | 264.33 ± 3.06 | 263.00 ± 5.57 | 259.83 ± 6.27 |
| Body Weight | | | | | |
| TC (mg/dl) | 134.33 ± 4.04 | 133.00 ± 4.58 | 129.33 ± 2.08 | 117.67 ± 6.66 | 116.49 ± 6.04 |
| TG (mg/dl) | 38.33 ± 2.52 | 37.50 ± 2.00 | 35.97 ± 0.74 | 34.53 ± 0.83 | 34.19 ± 0.83 |
| HDL-C | 41.67 ± 1.53 | 40.17 ± 0.58 | 41.23 ± 0.38 | 39.70 ± 0.46 | 38.94 ± 0.82 |
| (mg/dl) | | | | | |
| LDL-C | 90.67 ± 3.21 | 90.60 ± 3.55 | 85.03 ± 0.21 | 84.53 ± 0.83 | 83.69 ± 1.18 |
| (mg/dl) | | | | | |
| VLDL | 3.83 ± 0.38 | 3.91 ± 0.34 | 3.88 ± 0.04 | 3.69 ± 0.05 | 3.65 ± 0.06 |
| (mg/dl) | | | | | |
| WBC(103/μl) | 8.67 ± 0.59 | 8.57 ± 0.42 | 8.58 ± 0.04 | 8.51 ± 0.14 | 8.42 ± 0.16 |
| RBC(10⁶/μl) | 7.73 ± 0.52 | 7.97 ± 0.32 | 7.73 ± 0.03 | 7.65 ± 0.08 | 7.57 ± 0.11 |
| Hb (g/dl) | 12.53 ± 0.38 | 12.58 ± 0.08 | 12.62 ± 0.07 | 12.87 ± 0.26 | 12.74 ± 0.27 |
| PCV (%) | 41.43 ± 1.07 | 41.63 ± 1.02 | 41.64 ± 0.07 | 41.42 ± 0.50 | 41.01 ± 0.64 |
| Reticulocyte | 1.24 ± 0.04 | 1.26 ± 0.01 | 1.27 ± 0.02 | 1.28 ± 0.07 | 1.26 ± 0.06 |
| (%) | | | | | |
| Platelet | 1078.67 ± 11.02 | 1086.67 ± 8.02 | 1086.00 ± 8.19 | 1069.00 ± 18.73 | 1058.31 ± 20.31 |
| count | | | | | |
| 10⁵/mm³ | | | | | |
| Clotting time | 113.33 ± 1.53 | 113.50 ± 1.00 | 113.00 ± 0.70 | 113.17 ± 0.35 | 112.04 ± 1.28 |
| (Sec) | | | | | |
| Neutrophils | 25.87 | 25.97 | 26.1 | 25.9 | 25.81 |
| Lymphocyte | 72 | 72 | 71.3 | 71.58 | 71.70 |
| Monocyte | 1.35 | 1.35 | 1.34 | 1.27 | 1.25 |
| Eosinophil | 1.25 | 1.25 | 1.26 | 1.25 | 1.24 |

TABLE 11
Anthropometrical and Biochemical Data of Statin + HFD continued rats (Mean + S.D.)

| | Animal Group | | | | |
|---|---|---|---|---|---|
| | Statin + HFD (Continued) | | | | |
| | Days | | | | |
| Parameters | 0 | 15 | 30 | 45 | 60 |
| Average | 275.33 ± 7.09 | 270.67 ± 7.37 | 268.33 ± 2.52 | 265.67 ± 6.51 | 262.33 ± 7.15 |
| Body Weight | | | | | |
| TC (mg/dl) | 139.67 ± 4.04 | 138.00 ± 4.00 | 134.00 ± 3.00 | 126.33 ± 4.51 | 125.15 ± 4.20 |
| TG (mg/dl) | 38.67 ± 2.08 | 38.00 ± 1.80 | 37.30 ± 0.44 | 34.73 ± 1.37 | 34.41 ± 1.26 |
| HDL-C | 40.33 ± 2.08 | 40.83 ± 1.15 | 42.45 ± 0.44 | 40.20 ± 2.07 | 39.82 ± 1.88 |
| (mg/dl) | | | | | |
| LDL-C | 91.67 ± 3.06 | 92.83 ± 3.82 | 91.33 ± 0.21 | 90.37 ± 0.85 | 89.52 ± 1.20 |
| (mg/dl) | | | | | |
| VLDL | 3.87 ± 0.31 | 3.93 ± 0.20 | 3.91 ± 0.05 | 3.86 ± 0.11 | 3.82 ± 0.10 |
| (mg/dl) | | | | | |
| WBC(103/μl) | 8.45 ± 0.39 | 8.58 ± 0.32 | 8.72 ± 0.07 | 8.65 ± 0.08 | 8.57 ± 0.12 |
| RBC(10⁶/μl) | 7.93 ± 0.15 | 8.07 ± 0.15 | 7.95 ± 0.03 | 7.90 ± 0.05 | 7.83 ± 0.09 |
| Hb (g/dl) | 12.30 ± 0.20 | 12.40 ± 0.20 | 12.83 ± 0.25 | 12.88 ± 0.28 | 12.76 ± 0.28 |
| PCV (%) | 42.57 ± 1.36 | 43.43 ± 1.53 | 42.85 ± 0.25 | 42.93 ± 0.42 | 42.53 ± 0.57 |
| Reticulocyte | 1.28 ± 0.03 | 1.34 ± 0.03 | 1.31 ± 0.03 | 1.33 ± 0.02 | 1.32 ± 0.02 |
| (%) | | | | | |
| Platelet | 1071.00 ± 13.89 | 1071.67 ± 11.55 | 1087.00 ± 3.61 | 1079.00 ± 14.53 | 1068.89 ± 16.98 |
| count | | | | | |
| 10⁵/mm³ | | | | | |
| Clotting time | 113.33 ± 1.15 | 114.33 ± 1.15 | 114.37 ± 0.71 | 114.40 ± 0.46 | 113.33 ± 1.24 |
| (Sec) | | | | | |
| Neutrophils | 25.5 | 25.5 | 26 | 25.73 | 25.70 |
| Lymphocyte | 72 | 72 | 71.2 | 71.67 | 71.67 |
| Monocyte | 1.35 | 1.35 | 1.32 | 1.33 | 1.34 |
| Eosinophil | 1.25 | 1.25 | 1.28 | 1.27 | 1.28 |

TABLE 12
Anthropometrical and Biochemical Data of Composition of the present
invention + HFD discontinued rats (Mean ± S.D.).

| | Animal Group | | | | |
|---|---|---|---|---|---|
| | Composition of the present invention + HFD (Discontinued) | | | | |
| | Days | | | | |
| Parameters | 0 | 15 | 30 | 45 | 60 |
| Average | 273.00 ± 7.00 | 272.00 ± 7.81 | 259.38 ± 3.31 | 253.83 ± 4.71 | 245.00 ± 5.48 |
| Body Weight | | | | | |
| TC (mg/dl) | 137.00 ± 3.46 | 132.00 ± 2.00 | 123.33 ± 1.71 | 112.50 ± 5.79 | 107.29 ± 3.93 |
| TG (mg/dl) | 41.33 ± 3.21 | 40.67 ± 3.51 | 34.25 ± 0.34 | 31.48 ± 0.57 | 30.53 ± 0.55 |
| HDL-C | 42.00 ± 2.65 | 40.00 ± 1.00 | 41.09 ± 0.66 | 42.80 ± 0.31 | 44.15 ± 1.06 |
| (mg/dl) | | | | | |
| LDL-C | 92.00 ± 4.36 | 92.00 ± 3.61 | 83.28 ± 0.51 | 78.36 ± 0.40 | 76.01 ± 0.39 |
| (mg/dl) | | | | | |
| VLDL | 3.77 ± 0.55 | 3.77 ± 0.55 | 3.74 ± 0.05 | 3.72 ± 0.09 | 3.60 ± 0.09 |
| (mg/dl) | | | | | |
| WBC(103/μl) | 8.70 ± 0.40 | 8.77 ± 0.21 | 8.83 ± 0.03 | 8.27 ± 0.15 | 8.02 ± 0.15 |
| RBC(10⁶/μl) | 8.03 ± 0.40 | 8.10 ± 0.44 | 8.16 ± 0.04 | 7.56 ± 0.20 | 7.33 ± 0.19 |
| Hb (g/dl) | 12.53 ± 0.25 | 12.63 ± 0.25 | 12.76 ± 0.19 | 12.69 ± 0.34 | 11.66 ± 0.23 |
| PCV (%) | 42.63 ± 1.69 | 42.90 ± 1.28 | 43.05 ± 0.14 | 39.95 ± 0.94 | 38.75 ± 0.91 |
| Reticulocyte | 1.22 ± 0.10 | 1.25 ± 0.08 | 1.26 ± 0.02 | 1.19 ± 0.04 | 1.15 ± 0.04 |
| (%) | | | | | |
| Platelet | 1070.00 ± 13.23 | 1065.67 ± 13.50 | 1081.23 ± 10.09 | 1016.50 ± 17.92 | 1069.34 ± 15.08 |
| count | | | | | |
| 10⁵/mm³ | | | | | |
| Clotting time | 112.67 ± 2.52 | 113.67 ± 2.08 | 113.37 ± 0.62 | 107.27 ± 0.63 | 104.05 ± 0.61 |
| (Sec) | | | | | |
| Neutrophils | 25.5 | 25.93 | 25.9 | 26.65 | 26.85 |
| Lymphocyte | 72 | 71.47 | 71.5 | 70.7 | 70.58 |
| Monocyte | 1.35 | 1.33 | 1.31 | 1.33 | 1.29 |
| Eosinophil | 1.25 | 1.27 | 1.29 | 1.32 | 1.28 |

TABLE 12
Anthropometrical and Biochemical Data of Composition of the present
invention + HFD continued rats (Mean + S.D.)

| | Animal Group | | | | |
|---|---|---|---|---|---|
| | Composition of the present invention + HFD (Continued) | | | | |
| | Days | | | | |
| Parameters | 0 | 15 | 30 | 45 | 60 |
| Average | 271.00 ± 6.56 | 267.33 ± 6.43 | 261.60 ± 3.16 | 256.83 ± 5.04 | 248.83 ± 5.04 |
| Body Weight | | | | | |
| TC (mg/dl) | 140.00 ± 4.58 | 134.67 ± 3.06 | 133.80 ± 1.87 | 119.67 ± 4.68 | 117.27 ± 4.58 |
| TG (mg/dl) | 40.67 ± 3.06 | 38.67 ± 3.06 | 37.47 ± 0.81 | 33.13 ± 0.60 | 32.47 ± 0.59 |
| HDL-C | 40.33 ± 2.52 | 46.67 ± 3.79 | 45.91 ± 0.19 | 40.32 ± 0.55 | 39.51 ± 0.54 |
| (mg/dl) | | | | | |
| LDL-C | 92.33 ± 2.52 | 88.33 ± 3.06 | 88.19 ± 0.29 | 82.48 ± 0.43 | 80.83 ± 0.42 |
| (mg/dl) | | | | | |
| VLDL | 3.47 ± 0.35 | 3.40 ± 0.20 | 3.36 ± 0.03 | 3.64 ± 0.09 | 3.57 ± 0.09 |
| (mg/dl) | | | | | |
| WBC(103/μl) | 8.83 ± 0.35 | 9.07 ± 0.42 | 9.01 ± 0.05 | 8.70 ± 0.16 | 8.53 ± 0.16 |
| RBC(10⁶/μl) | 7.93 ± 0.45 | 8.27 ± 0.21 | 8.08 ± 0.14 | 7.95 ± 0.21 | 7.79 ± 0.21 |
| Hb (g/dl) | 12.30 ± 0.26 | 12.83 ± 0.31 | 12.92 ± 0.08 | 12.66 ± 0.25 | 12.41 ± 0.25 |
| PCV (%) | 40.10 ± 1.49 | 41.10 ± 0.56 | 41.44 ± 0.50 | 42.05 ± 0.99 | 41.21 ± 0.97 |
| Reticulocyte | 1.20 ± 0.05 | 1.26 ± 0.09 | 1.25 ± 0.03 | 1.25 ± 0.04 | 1.22 ± 0.04 |
| (%) | | | | | |
| Platelet | 1085.00 ± 13.23 | 1091.67 ± 6.51 | 1086.42 ± 6.08 | 1070.00 ± 18.87 | 1048.60 ± 18.49 |
| count | | | | | |
| 10⁵/mm³ | | | | | |
| Clotting time | 112.00 ± 2.00 | 112.33 ± 0.58 | 112.05 ± 0.91 | 112.92 ± 0.66 | 110.66 ± 0.65 |
| (Sec) | | | | | |
| Neutrophils | 25.5 | 25.5 | 26.1 | 26.03 | 25.50 |
| Lymphocyte | 72 | 72 | 71.3 | 71.39 | 71.96 |
| Monocyte | 1.35 | 1.35 | 1.3 | 1.33 | 1.30 |
| Eosinophil | 1.25 | 1.25 | 1.3 | 1.26 | 1.23 |

It can be inferred from table 7 to 12 that the reduction in total cholesterol, LDL and TG was significantly higher in the group that used the composition of the present invention, i.e. 22%, 17% and 26% in comparison to statin group 13%, 8% and 11% respectively. The level of HDL also improved in the group that used the composition of the present invention as compared with statin group i.e., 44 mg/dl vs 39 mg/dl.

TABLE 13
Biochemical Data and Organ Body weight after necropsy in all the groups in rats (Mean + S.D.).

| | | | | 3A. | 3B. | 4A. | 4B. |
|---|---|---|---|---|---|---|---|
| | 1. | | | Statin + | Statin + | Formulation + | Formulation + |
| | Normal | 2A. HFD | 2B. HFD | HFD | HFD | HFD | HFD |
| Parameters | Control | Discontinued | Continued | Discontinued | Continued | Discontinued | Continued |
| MDA Plasma | 34.51 ± 1.72 | 41.31 ± 1.91 | 45.38 ± 1.22 | 35.80 ± 1.58 | 35.84 ± 0.97 | 34.17 ± 1.41 | 36.88 ± 1.63 |
| (nmol/ml) | | | | | | | |
| MDA Liver | 79.33 ± 1.33 | 86.47 ± 2.33 | 98.51 ± 1.96 | 82.65 ± 3.76 | 84.53 ± 5.09 | 77.71 ± 1.61 | 81.53 ± 0.94 |
| (nmol/g tissue) | | | | | | | |
| HMG Co | 214.29 ± 3.89 | 255.34 ± 3.45 | 274.37 ± 3.55 | 170.03 ± 2.71 | 224.83 ± 18.48 | 167.71 ± 3.45 | 193.80 ± 7.36 |
| Reductase | | | | | | | |
| (U/mg) | | | | | | | |
| Creatine | 1.26 ± 0.03 | 1.32 ± 0.02 | 1.38 ± 0.03 | 1.32 ± 0.08 | 1.31 ± 0.08 | 1.24 ± 0.03 | 1.29 ± 0.05 |
| phosphokinase | | | | | | | |
| LDH (U/ml) | 285.37 ± 4.23 | 315.35 ± 5.96 | 328.52 ± 5.13 | 284.07 ± 5.61 | 288.32 ± 12.64 | 284.24 ± 3.80 | 292.59 ± 5.78 |
| SGOT (U/ml) | 59.75 ± 1.25 | 73.53 ± 0.99 | 77.88 ± 2.93 | 62.88 ± 3.43 | 63.76 ± 2.92 | 57.65 ± 1.77 | 62.02 ± 1.56 |
| SGPT (U/ml) | 53.57 ± 1.23 | 67.75 ± 1.09 | 71.69 ± 1.84 | 64.69 ± 0.52 | 53.82 ± 1.13 | 52.53 ± 1.55 | 54.22 ± 0.53 |
| HbA1c | 3.76 ± 0.17 | 4.76 ± 0.11 | 5.11 ± 0.18 | 4.15 ± 0.16 | 3.67 ± 0.18 | 3.68 ± 0.16 | 3.83 ± 0.16 |
| Liver (g/ | 3.40 ± 0.12 | 5.1 ± 0.13 | 4.31 ± 0.09 | 4.37 ± 0.05 | 4.51 ± 0.12 | 3.41 ± 0.12 | 3.40 ± 0.05 |
| 100 g) | | | | | | | |
| Brain (g/ | 3.40 ± 0.12 | 3.97 ± 0.13 | 4.31 ± 0.09 | 3.37 ± 0.05 | 3.51 ± 0.12 | 3.41 ± 0.12 | 3.40 ± 0.05 |
| 100 g bd. wt) | | | | | | | |
| Heart (g/ | 1.37 ± 0.04 | 1.39 ± 0.03 | 1.43 ± 0.02 | 1.32 ± 0.05 | 1.34 ± 0.02 | 1.37 ± 0.03 | 1.34 ± 0.05 |
| 100 g bd. wt) | | | | | | | |
| Kidney (g/ | 2.85 ± 0.04 | 3.01 ± 0.06 | 3.15 ± 0.06 | 2.84 ± 0.04 | 2.82 ± 0.06 | 2.83 ± 0.03 | 2.87 ± 0.04 |
| 100 g bd. wt) | | | | | | | |
| Spleen | 1.16 ± 0.03 | 1.18 ± 0.01 | 1.21 ± 0.03 | 1.17 ± 0.03 | 1.19 ± 0.02 | 1.17 ± 0.04 | 1.18 ± 0.03 |
| Lungs (g/ | 0.64 ± 0.02 | 0.68 ± 0.01 | 0.72 ± 0.02 | 0.66 ± 0.03 | 0.65 ± 0.03 | 0.64 ± 0.03 | 0.67 ± 0.03 |
| 100 g bd. wt) | | | | | | | |
| Intestine | 2.01 ± 0.04 | 2.02 ± 0.02 | 2.14 ± 0.04 | 2.00 ± 0.02 | 2.00 ± 0.02 | 1.99 ± 0.03 | 2.02 ± 0.02 |
| Pancrease (g/ | 0.88 ± 0.01 | 0.92 ± 0.04 | 0.91 ± 0.05 | 0.91 ± 0.03 | 0.89 ± 0.05 | 0.92 ± 0.04 | 0.93 ± 0.03 |
| 100 g bd. wt) | | | | | | | |
| 1,25- | 43.00 | 39.00 | 24.00 | 35.00 | 34.00 | 42.00 | 37.00 |
| dihydroxy- | | | | | | | |
| cholecalciferol | | | | | | | |
| (ng/ml) | | | | | | | |
| Ca (mg/dl) | 10.20 | 11.00 | 13.80 | 12.10 | 12.80 | 11.90 | 11.20 |
| Na (mmol/l) | 141.00 | 147.00 | 167.00 | 148.00 | 154.00 | 141.00 | 145.00 |
| K (mmol/l) | 4.67 | 5.20 | 7.80 | 6.20 | 6.11 | 4.75 | 6.23 |

It is evident from table no 11 that Liver LPO reduced by the composition of the present invention was 18%, whereas with statin it was 11%. Inhibition of HMG Co reductase activity by the composition of the present invention and statin was found to be similar i.e., 35%. The SGOT reduction by the composition of the present invention was high at 22% as compared to statin at 16%. Serum glutamic pyruvic transaminase (SGPT) reduction was significantly high by the composition of the present invention i.e., 23% as compared to statin i.e., 5%.

### Example 4: Histopathological Assay on Aorta

FIG. 6 and FIG. 12 depicts the histopathology of Aorta rats. The histopathological findings in the Aorta of High Fat Diet Discontinued plaque was found indicating the successful development of atherosclerotic rat model and more severe plaque formation was observed in HFD continued group. The intervention with the statin with HFD discontinued showed a moderate reduction in the plaque while, in statin with HFD continued it was mild. The morphology in the groups taking the composition of the present invention indicated the complete plaque reduction/dissolution almost similar to normal rat aorta.
The Examples 1 to 4 depict that in the prophylactic study which are summarized hereinabove in the mean and standard deviation of lipid profile and hematological parameters indicated that most significant findings that the composition of the present invention was able to maintain the body weight even after HFD treatment, whereas statin was not able to maintain the body weight. Further even though the increase in TC was similar (10%) in both the composition of the present invention and statin groups even after HFD diet treatment, it is evident the HFD treatment induced the lipid peroxidation in liver by 20% which was significantly reduced (23%) by the group provided with the composition of the present invention (twice daily) and 15% by statin treatment. Creatine phosphokinase activity was also found high in both HFD, and statin treated groups (8%-10%) which indicated the muscular damage whereas no muscular damage reported in the group provided with the composition of the present invention.
Moreover, the inhibition in SGOT (24%) and SGPT (29%) activity was also found maximum in the group treated with the composition of the present invention (twice daily) where others group failed to manage the level of SGPT and SGOT. However, both the composition of the present invention and statin treatment was able to maintain Blood glucose level similar to normal diet group even after HFD fed groups.
Further, the histopathological findings of prophylactic study in the heart showed a clear deposition of fat in the cardiac muscles in HFD induced rats as compared to normal rat heart. While in case of statin the heart muscle showed relaxation in the cardiac muscles as the fat content was decreased. Moreover, to this the rats treated with the composition of the present invention showed improved or much better recovery in cardiac muscle morphology even during the treatment with HFD. The histopathological finding of liver showed a significant formation fatty cells (grade 2) in HFD induced rats, however the intervention of statin and the composition of the present invention at OD and BD dose levels showed decreasing order of fatty liver from grade 2 to grade 1 (evidenced by dilation in hepatic portal triad). In all other groups no significant changes were observed. Statistically the data showed significant association in biochemical and clinical profiling from day 0 to day 90 (P<0.001). These examples depict that the composition of the present invention has significant cholesterol lowering effect on hypercholesterolemia induced wistar rat's models even in the group which was treated with continuous high fat diet and showed improved results as compared to the statin (Atorvastatin).
The Examples 1 to 4 depict that in the therapeutic study, reduction in total cholesterol, LDL and TG was significantly higher in group treated with the composition of the present invention i.e., 22%, 17% and 26% in comparison to statin group 13%, 8% and 11% respectively. Further, the anthropometrical and biochemical data of normal control rats showed normal with the increase in 10% body weight during the whole in house study of 60 days. In High Fat Diet (HFD) Continued group the body weight and lipid profile parameters showed significant increase in the visceral mass and are very lethargic as compared to that of normal healthy controls.
Moreover, the inhibition of HMG Co reductase activity by the composition of the present invention and statin was found to be similar i.e., 35%. The SGOT reduction was high 22% as compared to statin 16%. SGPT reduction was significantly high in group treated with the composition of the present invention i.e., 23% as compared to statin group i.e., 5%. These examples depict that the composition of the present invention has significant effect on atherosclerosis and has anti-hypercholesterolemia effect on hypercholesterolemia induced wistar rats' models.
The examples illustrate that the composition of the present invention at 135 mg/Kg body weight (BD) oral dosage showed no sign of toxicity and no mortality and was found to be safe as per animal in house milieu. The Blood parameter/markers were correlated with the histopathological analysis as mentioned below: The histopathological finding of liver showed a significant-formation of fatty cells (grade 3) in HFD induced rats (continued), however the intervention of statin and the composition of the present invention showed more recovery in group provided with the composition of the present invention (evidenced by dilation in hepatic portal triad) in comparison to the statin group. Moreover, the group provided with the composition of the present invention improved the histopathology of fatty liver from grade 3 to grade 1. In Histopathological findings in the Aorta of High Fat Diet Discontinued plaque was found indicating the successful development of atherosclerotic rat model and more severe plaque formation was observed in HFD continued group. The intervention with the statin with HFD discontinued showed a moderate reduction in the plaque while, in statin with HFD continued it was mild. The morphology in the groups provided with the composition of the present invention indicated the complete plaque reduction/dissolution almost similar to normal rat aorta. The histopathological image also indicated the plaque development post high fat diet treatment. The similar plaque was observed to clear/dissolve completely post treatment with the composition of the present invention.
In histopathology study, normal group showed no pathological changes in endothelial lining. Control group (High cholesterol diet) showed severe damage to endothelial lining. The composition of the present invention treated groups showed less damage to the endothelial lining as compared to HFD group while atorvastatin treated group showed mild damage to the endothelial lining. Atherosclerosis was induced in HFD group and the composition of the present invention restored the normal state as shown in histological studies. Moreover, the deficiency of Vitamin D was also associated with the treatment of atorvastatin (35 IU) whereas the composition of the present invention (44 IU) maintained the healthy vitamin D value in the plasma as compared with control (42 IU). Thereby correlating the side effects associated with a statin regular treatment of 60 days.
Further, it is evident that Liver LPO reduced by the composition of the present invention was 18%, whereas with statin it was 11%. Inhibition of HMG Co reductase activity by the composition of the present invention and statin was found to be similar i.e., 35%. The SGOT reduction was high 22% as compared to statin 16%. SGPT reduction was significantly high in the group provided with the composition of the present invention i.e., 23% as compared to the statin group i.e., 5%.
Moreover, the examples also highlight liver damage/fatty liver associated with statin regular treatment correlated with histopathological analysis. The CPK value was found to be increased by 10% in the statin group which reflects the muscular damage. The composition of the present invention does not increase the level of CPK. The muscular damage is also associated with pain such as muscular fatigue caused by the statin treatment. Sodium and potassium were also found enhanced in the statin group whereas the composition of the present invention maintained the level of Na (Sodium) and K (Potassium). The long-term rise in the electrolyte such as Na and are associated with blood pressure disorders impacting the heart. Thus, the composition of the present invention showed more efficacy for its anti-atherosclerotic and anti-hypercholesteremic potential in comparison to statin and for both the prophylactic and therapeutic studies at the concentration of 82.5 mg/Kg body weight (OD & BD) and 135 mg/Kg body weight (BD) of the composition of the present invention at oral dosage did not show any sign of toxicity and mortality and was thus found to be safe. Moreover, the Histopathology and Blood markers studies indicate that the side effects of statin are not associated with long term use of the composition of the present invention such as Vitamin D deficiency, Liver damage, Na and K, and muscular fatigue.

While the present invention has been described with respect to certain embodiments, it will be apparent to those skilled in the art that various changes and modification may be made without departing from the scope of the invention as defined in the following claims.

1. An anti-hypercholesterolemia composition comprising:
Basella alba leaf extract in the range of 30 to 70 wt %;
red yeast rice extract in the range of 5 to 50 wt %;
squalene extract in the range of 1 to 30 wt %;
Cordyceps extract in the range of 10 to 70 wt %; and
at least one nutraceutically or pharmaceutically acceptable excipient in the range of 0.01% to 50%.

2. The composition as claimed in claim 1, wherein said at least one nutraceutically or pharmaceutically acceptable excipient is at least one selected from the group consisting of diluents, super disintegrants, binders, lubricants, glidants, fillers, vitamins, minerals, phytochemicals and antioxidants.

3. The composition as claimed in claim 2, wherein said at least one binder is at least one selected from the group consisting of Gelatin, ethyl cellulose, starch, Poly vinyl Pyrrolidone, Sodium alginate, carboxy methyl cellulose, Silicon oxide, Neusilin US2, Dextrin, Talc, Magnesium stearate, Aerosil and Micro crystalline cellulose.

4. The composition as claimed in claim 2, wherein said at least one lubricant is at last one selected from the group consisting of Stearic acid, Calcium stearate, Sodium Benzoate and Polyethylene glycol.

5. The composition as claimed in claim 2, wherein said at least one glidant is at least one selected from the group of Corn starch and Silicon dioxide.

6. The composition as claimed in claim 2, wherein said at least one super disintegrant is at least one selected from the group consisting of Lactose, Micro crystalline cellulose and sorbitol.

7. The composition as claimed in claim 2, wherein said:
vitamin is one selected from the group consisting of vitamin D, vitamin C, vitamin E, vitamin A and vitamin K;
mineral is one selected from the group consisting of Zinc, Calcium, Magnesium, iron and selenium;
phytochemical is curcumin; and
antioxidant is one selected from the group consisting of quercetin and resveratrol.

8. The composition as claimed in claim 1, wherein said Basella alba extract comprises:
polyphenols in an amount in the range of 0.5% to 2%;
phenolics in an amount in the range of 0.5% to 2%;
flavonoids in the range of 0.2% to 3%; and
ascorbic acid in the range of 0.2% to 1%.

9. The composition as claimed in claim 1, wherein the Monacolin K content in said red yeast rice extract is 0.1 ppm to 100 ppm and the Ankaflavin content in said red yeast rice extract is 0.2% to 5%.

10. A method for manufacturing an anti-hypercholesterolemia composition, said process comprises:
preparing the extracts of Basella alba leaf extract, Cordyceps extract, red yeast rice extract and squalene extract;
wherein said Basella alba leaf extract is prepared by:
drying a pre-determined amount of the Basella alba leaves followed by milling to obtain a milled plant material having a desired particle size;
extracting the milled plant material using solvents selected from the group of Ethanol, Hexane, Acetone, Double distilled water; followed by removal and recovery of the solvent;
nitrogen flushing for removal of the residual traces of the solvent to obtain a crude extract;
winterizing the crude extract by dissolving the crude extract in at least one of ethanol, methanol and hexane followed by deep freezing;
cold filtering for the removal of the unwanted waxy material followed by addition of pre-determined amounts of Aerosil and Dextrin to the crude extract to obtain the Basella alba leaf extract;
wherein said Cordyceps extract is prepared by:
growing a pre-determined amount of the Cordyceps culture in the form of mycelium or fruiting body; and
collecting and drying the fruiting bodies or mycelium followed by powdering to obtain the Cordyceps extract;
wherein said red yeast rice extract is prepared by:
grinding the red yeast rice to obtain a powder;
pulverizing or homogenizing using solvents selected from the group of water, ethanol, acetone, methanol, Acetone, Di-ethyl ether and hexane followed by filtration to obtain an extract;
keeping the extract undisturbed for 8 to 12 hours to obtain a red precipitate and a yellow supernatant;
subjecting the yellow supernatant to drying to obtain a slurry;
adding drying excipients to the slurry to obtain the extract of red yeast rice in powder form; and
blending together the extracts and pre-determined amounts of at least one nutraceutically or pharmaceutically acceptable excipient to obtain the anti-hypercholesterolemia composition.

11. An anti-hypercholesterolemia composition for the treatment or prevention of hypercholesterolemia and arteriosclerosis comprising:
Basella alba leaf extract in the range of 30 to 70 wt %;
red yeast rice extract in the range of 5 to 50 wt %;
squalene extract in the range of 1 to 30 wt %;
Cordyceps extract in the range of 10 to 70 wt %; and
at least one nutraceutically or pharmaceutically acceptable excipient in the range of 0.01 to 50 wt %.

12. The composition as claimed in claim 11, wherein said at least one nutraceutically or pharmaceutically acceptable excipient is one selected from the group consisting of diluents, super disintegrants, binders, lubricants, glidants, fillers, vitamins, minerals, phytochemicals and antioxidants.
